(19) United States
(12) Patent Application Publication
(10) Pub. No.: US 2005/0277125 A1
(43) Pub. Date: Dec. 15, 2005

(54) High-density reaction chambers and methods of use

(75) Inventors: James A. Benn, Arlington, MA (US); Mats Cooper, Chicago, IL (US); Todd Thorsen, Arlington, MA (US)

Correspondence Address: Patrick R.H. Waller, Ph.D.;Wolf, Greenfield & Sacks, P.C., 600 Atlantic Avenue, Boston, MA, 02210, US

(73) Assignee: Massachusetts Institute of Technology, Cambridge, MA (US)

(21) Appl. No.: 10/976,168

(22) Filed: Oct. 27, 2004

Related U.S. Application Data
(60) Provisional application No. 60/514,887, filed on Oct. 27, 2003.

(51) Int. Cl. C12Q 1/68; C12M 1/34

(52) U.S. Cl. 435/6; 435/287.2

(57) ABSTRACT

Methods and devices for performing multiple simultaneous reactions on a reaction surface are disclosed. Methods and devices for simultaneously interrogating multiple patient samples with multiple diagnostic reagents are disclosed.

### RELATED APPLICATION

This application claims benefit under 35 U.S.C. 119(e) of U.S. provisional patent application 60/514,887, filed Oct. 27, 2003, the entire content of which is incorporated herein by reference.

### BACKGROUND OF INVENTION

1. Field of Invention

The invention generally relates to methods and devices for performing high throughput biological assays. In particular, the invention relates to microarray methods and devices for nucleic acid diagnostic assays.

2. Discussion of Related Art

There is an ongoing need to reduce the cost of molecular assays for both research and clinical applications. Research efforts that use population studies to identify associations between different diseases and genetic characteristics are limited by the cost of molecular testing. The use of routine genetic testing in clinical settings also has been limited by the high cost of individual assays. Nonetheless, an increasing number of genetic targets have been shown to have clinical significance, either for the prediction of disease risk factors or for the early-stage detection of disease. Therefore, there is a pressing need for low-cost methodologies that can be implemented effectively to make these genetic targets available for routine clinical use.

Most molecular assays for determining the presence and/or quantity of particular biological molecules involve detecting the binding of specific reagents to biological molecules such as nucleic acids or proteins that are present in a sample. For example, the presence of a particular DNA molecule in a sample is typically detected using an assay that involves hybridizing a probe to the DNA molecule. In assays where several targets are being simultaneously tested for, the general approach involves immobilizing one group of reactants, labeling a second group of reactants, and then exposing the labeled reactants to the immobilized reactants. The immobilized reactants are then queried to determine whether any of the labeled reactants were bound to them. For example, a Dot Blot DNA assay involves immobilizing sample DNA on a flat surface and exposing labeled nucleic acid probes to the immobilized DNA. In contrast, a reverse Dot Blot assay involves immobilizing nucleic acid probes on a flat surface and exposing labeled sample DNA to the immobilized probes. Many commercially-available DNA tests use reverse Dot Blot configurations. A customer may purchase a glass slide that has different classes of probe DNA attached to it. The customer may then label sample DNA, expose it to the glass slide, and query the slide for the presence of label indicative of hybridized sample DNA. The presence of a statistically significant amount of hybridized sample DNA at a particular position (and also at duplicate positions) on the slide is indicative of the presence, in the sample DNA, of one or more sequences complementary to the probe that is attached to the glass slide at that position.

The costs of molecular assays have been reduced over the past few years through increased parallelism and miniaturization of hybridization assays. The development of microarrays has increased the ability to perform parallel tests of many targets in a single sample, thereby reducing both labor and reagent costs. DNA microarrays typically include a predefined pattern of many different DNA molecules bound to a flat surface. This pattern typically consists of spots of DNA that range from 60 to 150 microns in diameter, spaced 250 to 350 microns apart, resulting in approximately 4000 spots per square centimeter. Even though a glass slide may contain 50,000 or more DNA spots, there are typically 5 duplicates for each spot so that only about 10,000 different DNA groups are represented on the slide. Microarray hybridization may be performed by exposing all the DNA groups arrayed on the flat surface to a single sample of labeled DNA fragments. Hybridization of the labeled fragments to the arrayed DNA is then measured in order to determine whether any of the labeled fragments were complementary to any of the arrayed DNA. In this configuration, only one or two different labels may be used for pooled samples of DNA fragments, because of the difficulty in discriminating between more than a couple of different labeled DNA molecules hybridized to a single spot on the flat surface. In this regard, this technology may not be used effectively for simultaneously testing many samples, such as from multiple patients, on a single microarray.

Attempts have been made to increase the number of patient samples that can be assayed on a single microarray platform. Modest improvements were achieved by breaking the microarray surface into different sections, each of which was then exposed to different samples for hybridization. In one effort, it was shown that 250 hybridization elements could be placed at the bottom of the wells of a standard 96-well microtiter plate (also referred to herein as a multi-well plate), thereby enabling a parallel analysis of 250 targets in 96 separate samples. This microtiter plate approach allowed standard automation procedures to be used for loading and processing of the microarray surfaces. However, this approach could not be scaled up to process higher numbers of samples and targets. For example, a standard 384-well microtiter plate, which has the same overall dimensions as a 96 well plate, provides only a quarter of the bottom surface area available in each well, and therefore only supports approximately 60 targets in each well.

Bead-based systems also have been used in attempts to increase sample throughput. By replacing the flat microarray surface with a bead surface, the surface area available for individual hybridizations is increased, thereby enabling parallel processing of an increased number of samples. DNA probes are attached to the surfaces of the beads, and labeled segments of sample DNA are exposed to the probes. The beads are then queried for the presence of label, which would indicate the hybridization of a labeled DNA fragment to a bead. This assay can be readily automated. However, each target in a sample must be individually amplified and labeled in order to produce the many individual segments of target DNA used for the hybridization assay. The process of amplifying and labeling individual segments is expensive and complicates the reuse of sample DNA for subsequent testing on different targets. Also, bead-based systems involve high sample volumes, bead counting, expensive equipment, and are limited to a small number of targets per sample. Therefore, despite the advantages of this procedure, there is still a pressing need in the art for methods and devices for performing multiple simultaneous assays on multiple samples.

### SUMMARY OF INVENTION

Aspects of the invention provide methods and devices for combining multiple samples and reagents in simultaneous parallel reactions. In one embodiment, these reactions may be performed using very small amounts of sample and reagent. In aspects of the invention, reduced amounts of sample and reagent manipulation steps may be used to set up a large number of reactions.

In one aspect, the invention relates to a method of forming a line of sample on a surface by (a) forming a contact between a reaction surface on a reaction substrate and an open microfluidic channel on a channel substrate; (b) introducing a sample solution into the microfluidic channel, wherein the sample solution contacts the reaction surface along a contact line formed by the contact between the reaction surface and the open microfluidic channel; and (c) disrupting the contact between the reaction surface and the microfluidic channel, thereby forming a line of sample on the reaction surface, wherein the line corresponds to the contact line.

In another aspect, the invention relates to a reaction surface array having a plurality of lines of immobilized reactant, wherein the array of immobilized reactants is produced using a method of the invention. The immobilized reactant may be a component of a sample such as a biological sample. Alternatively, the immobilized reagent may be a component of a reagent such as an oligonucleotide probe. The array of reactants may have a density of at least 50 sample lines per linear centimeter on the reaction surface.

In another aspect, the invention relates to method of contacting each member of a plurality of immobilized reactants with each member of a plurality of mobile reactants by (a) forming a contact between a reaction surface on a reaction substrate and an array of open microfluidic channels on a channel substrate, wherein the reaction surface comprises an array of lines of immobilized reactants; and, (b) introducing mobile reactant solutions into the microfluidic channels to form contacts between each of the immobilized and mobile reactants by intersecting each line of the immobilized reactant with a line of mobile reactant.

In another aspect, the invention relates to a method of connecting a matrix of sample wells to an array of microfluidic channels, by (a) contacting a first surface of a matrix of sample wells to a first surface of a transfer plate in order to form fluid connections between wells in the matrix and channels on the transfer plate; and, (b) contacting a second surface of the transfer plate to a first surface of an array of microfluidic channels in order to form fluid connections between microfluidic channels of the array and channels on the transfer plate.

In another aspect, the invention relates to an interface including a guide adapted to align a tip of a pipette toward an orifice within a guide of a guide plate and a retainer adapted to hold the tip of the pipette in the orifice. The guide may be a well in a multi-well plate having one or more orifices toward the bottom of each well. The guide may be shaped like a funnel. The retainer may be made of a compliant material that is adapted to conform to the pipette tip. The retainer also may form a seal. The retainer may be made of silicone or other compliant material. The retainer may be held within a groove of the guide.

In another aspect, the invention relates to a method of delivering solution from a pipette tip onto a channel or hole in a substrate such as an array of microfluidic channels or a transfer plate as described herein. The solution may be delivered by applying positive pressure to the pipette tip (e.g., by using a pipettor). Alternatively, the solution may be delivered by applying a vacuum to the channel or hole and drawing the solution out of the tip.

In another aspect, the invention relates to an apparatus including an array of open microfluidic channels each having a width of less than 500 microns. In one aspect, the invention relates, to an apparatus including an array of open microfluidic channels each having a depth of less than 500 microns.

In one embodiment, one or more channels in an array may be in fluid communication with one another in order to introduce a common sample onto the reaction surface. An array of microfluidic channels may be made of PDMS or other material.

One aspect of the invention includes depositing a first set of samples or reagents in a predetermined pattern on a solid substrate, and contacting the deposited material with a second set of samples or reagents to form a predetermined matrix of contact points between each of the samples and reagents. The contact points may then be observed to detect reactions or reaction products indicative of an interaction between one or more of the first set of reagents and one or more of the second set of reagents.

Another aspect of the invention provides methods and devices for depositing samples or reagent on the reaction substrate.

In another aspect, the invention provides methods and devices for contacting one or more reagents to one or more samples previously deposited or immobilized on a reaction surface. Still, according to another aspect the invention provides method and devices for contacting one or more samples to reagents that are immobilized on a reaction surface.

In yet another aspect of the invention, a combination microtiter plate and microfluidics device is provided that is useful to perform genetic tests on a series of patient samples simultaneously, which may reduce the cost and increase the speed of genetic testing. In another embodiment, the invention provides a device that enables samples to be reused for new genetic tests. In another embodiment, the invention provides a device that can be scaled up to perform many tests on many samples simultaneously. In another embodiment, the invention provides a device where the reaction kinetics of the tests can be optimized to achieve maximum accuracy, while using the lowest quantities of sample. In yet another embodiment, the invention provides a device that can be loaded with samples, reagents, and probes using standard inexpensive automation components.

In these and other aspects of the invention, a sample may be introduced at one end of the microfluidic channel and drawn into the microfluidic channel by applying a negative pressure to another end of the microfluidic channel. A contact line of the microfluidic channel may be straight, curved or of another shape. A contact line may be between 1 micron and 500 microns wide. A contact line of the microfluidic channel may be between 1 and 500 microns deep. In other embodiments, the height and depth may be different. A reaction substrate may be a glass plate. A channel array substrate may be PDMS. Each microfluidic channel in an array may have similar dimensions. A microfluidic channels may be separated by a channel walls each having a thickness of between 5 and 50 microns. A number of channels may be between 10 and 2,000.

These and other aspects of the invention are described in the following detailed description, examples, and attached figures. The examples and figures are non-limiting and other aspects of the invention will be apparent to one of skill in the art based on the detailed description, summary, and attached claims.

The disclosure of the scientific publications and patents listed herein are incorporated herein by reference in their entirety.

### BRIEF DESCRIPTION OF DRAWINGS

The accompanying drawings, are not intended to be drawn to scale. For purposes of clarity, not every component may be labeled in every drawing. In the drawings:

FIG. 1 shows flowchart of an assay according to one embodiment;

FIG. 2 shows an embodiment of step 110 of FIG. 1;

FIG. 3 shows an embodiment of contact step 1201;

FIG. 4 shows a contact points formed between at the intersections of immobilized rows of sample and reagents;

FIG. 5a shows a portion of an array of microfluidic channels;

FIG. 5b shows an array of microfluidic channels mated with a reaction substrate;

FIG. 6 shows an upper perspective view of a multi-well assembly;

FIG. 7 shows a lower perspective view of a multi-well assembly;

FIG. 8 shows a top view of a multi-well plate having apertures disposed in the bottom of each well;

FIG. 9 shows a top transparent view of a transfer plate mated with a microfluidic array;

FIG. 10 shows a top view of the transfer plate depicted in FIG. 9;

FIG. 11 shows a top view of the microfluidic array depicted in FIG. 9;

FIG. 12 shows a top view of another transfer plate mated with another microfluidic array;

FIG. 13 shows a top view of the transfer plate depicted in FIG. 12;

FIG. 14 shows a top view of the microfluidic array depicted in FIG. 12;

FIG. 15 shows a cross-section of a combination microfluidic array containing two channels that cross each other at right angles and communicate with each other;

FIG. 16 shows channels in a microfluidic array that can be opened to allow flow or closed to prevent flow or diffusion;

FIG. 17 shows droplets of reactant immobilized onto a reaction substrate;

FIG. 18 shows an image of target DNA deposited in lines on a reaction substrate;

FIG. 19 shows images of vertical lines of immobilized target DNA exposed to horizontal lines of labeled probe;

FIG. 20 shows a microfluidic device with 96 channels connected to entry ports;

FIG. 21 shows a single microfluidic channel having a serpentine configuration that is adapted to deliver a single reactant over multiple portions of a reaction substrate;

FIG. 22 shows a cross-sectional view of a microfluidic channel, according to one embodiment;

FIG. 23 shows a cross sectional view of a docking interface, according to one embodiment;

FIG. 24 shows a top view of a guide plate, as used with one embodiment of a docking interface;

FIG. 25 shows a docking interface placed within a clamping fixture;

FIG. 26 shows an array of microchannels that may be used with the docking interface shown in FIG. 25 as print channels;

FIG. 27 shows an array of microchannels that may be used with the docking interface shown in FIG. 25 as hybridization channels;

FIG. 28 shows an overlapped view of the arrays of microchannels shown in FIGS. 26 and 27;

FIG. 29 shows an top view of an array of 96 microchannels connected to inlet ports shown as cruciforms;

FIG. 30 shows an top view of exemplary components of devices according to the invention; 30a shows a transfer plate; 30b shows a channel array; 30c shows a channel array with a different channel configuration; 30d illustrates the overlay of the channel arrays of 30b and 30c;

FIG. 31 shows a cross section of a single channel on a reaction surface; the channel is mated to a transfer plate that in turn is mated to a reservoir support.

### DETAILED DESCRIPTION

Aspects of the invention relate to methods and devices for delivering material to a reaction site. A material may be a solution containing a reactant, e.g., a sample solution or a reagent solution. In one embodiment, aspects of the invention relate to methods and devices for delivering a first material to and/or depositing the first material on a surface where the material may be contacted with a second material. In one embodiment, aspects of the invention relate to methods and devices for delivering a first material to and/or depositing the first material on a surface where a second material has already been deposited. In one embodiment, aspects of the invention relate to methods and devices for delivering a material to a microfluidic channel or conduit.

Aspects of the invention provide an efficient approach to performing large numbers of reactions between multiple samples and/or reagents. Aspects of the invention may be useful for medical research and diagnostic procedures that involve running multiple tests on large numbers of patient samples. Aspects of the invention also may be useful for other applications that require mixing large numbers of samples and reagents in individual reactions. Aspects of the invention may be particularly useful for performing large numbers of reactions using small volumes of sample and reagent while minimizing the number of physical manipulations required to mix the samples and reagents. It should be appreciated that other aspects of the invention may be used for different applications as described herein.

Aspects of the invention relate to analytical methods and devices that are useful for a) depositing and/or immobilizing one or more reactants on a substrate, and/or b) contacting one or more deposited or immobilized reactants with one or more mobile reactant solutions. The contact points between the different reactants may be monitored for a reaction or signal of interest. Accordingly, the invention may be useful for conducting multiple simultaneous reactions where multiple test samples are individually exposed to various different reaction conditions or reagents. These aspects may be particularly useful for high throughput screening assays such as nucleic acid-based diagnostic assays. However, other medical assays or chemical reactions also can be performed and/or monitored, as the invention is not limited in this respect. Still, according to other aspects, devices may be used to provide sample to microarrays that have probe DNA immobilized thereon.

Other aspects of the invention relate to improved methods of providing reactants to a microarray, either for deposition onto the microarray or for contacting reactants previously deposited on the microarray. In some embodiments, a transfer plate may provide fluid connections between a multi-well plate or other macro-scale device (e.g., multi-pipettor, etc.) and an array of microfluidic channels. Still in some embodiments, a docking device or interface may assist sample delivery equipment, such as a pipette or multi-pipette (including one-dimensional and two-dimensional multi-pipettors) in interfacing with a transfer plate or an array of microfluidic channels such that a small volume of reactant may be efficiently delivered.

Other aspects of the invention relate to methods and devices for reducing the reaction time between a reagent and a sample. In one embodiment, very short hydridization times may be used to hybridize reagents and samples at interaction points using methods and devices of the invention. Parameters may be established to decrease reaction times in order to reduce overall assay times.

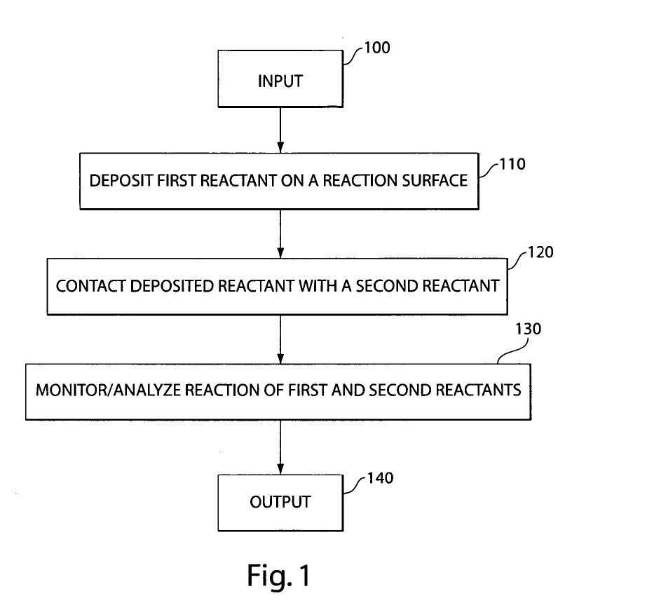

The block diagram of FIG. 1 shows an embodiment of the invention where a first reactant is deposited onto a reaction substrate 50 in act 110. The deposited reactant is contacted with a second reactant in act 120. The reaction between the first and second reactants is determined in act 130. The input reactants of act 100 (e.g., target nucleic acids in patient samples and/or diagnostic oligonucleotides in reagent solutions) will depend on the application chosen by the operator. Similarly, the output conclusions of act 140 (e.g. patient diagnosis or prognosis) will depend on the assays being performed and on the results of the assays. Other aspects of the invention may include fewer acts, additional acts, or alternative acts as described herein.

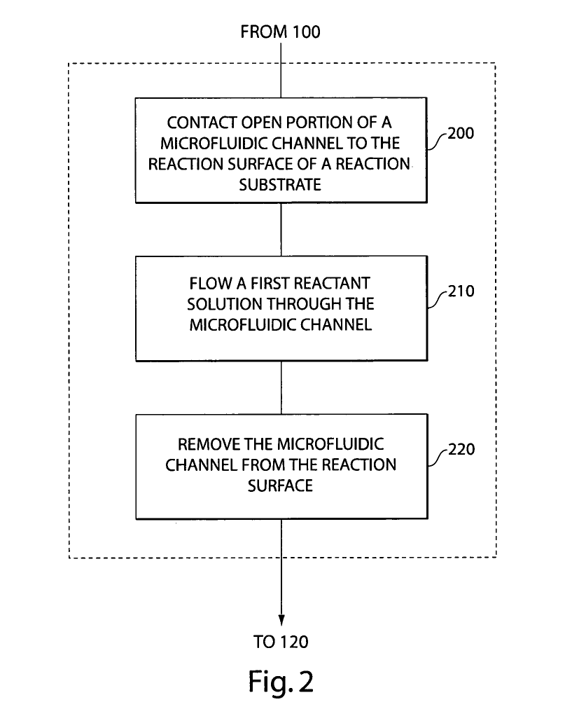

In one aspect, the invention relates to depositing at least one reactant on a substrate as exemplified by the block diagram of FIG. 2, which represents an embodiment of deposition act 110 from FIG. 1. In act 200 of FIG. 2, the open side 52 of an open microfluidic channel 54 is exposed to a reaction surface of a reaction substrate (e.g. a glass plate). The walls 56 of the open channel are contacted to the reaction surface to form a conduit or closed microfluidic channel 60 along the length of the interface between the open channel and the reaction substrate. In act 210, a volume of reactant solution is flowed into and/or through the microfluidic conduit to deposit reactant on the reaction surface. In act 220, the microfluidic channel is removed, and a trail or line 58 of reactant remains on the reaction surface. Other aspects of the deposition procedure may include fewer acts, additional acts, or alternative acts as described herein. It should be appreciated that an array of open microfluidic channels may be used to deposit a plurality of reactants onto a substrate.

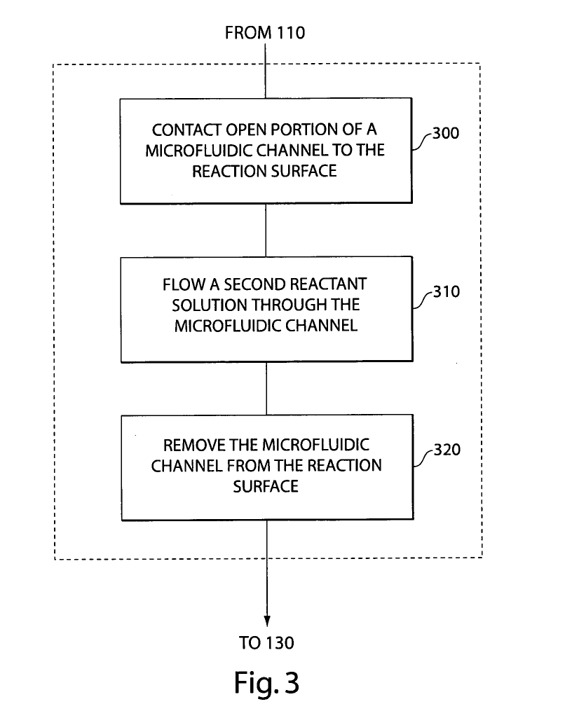

In another aspect, the invention relates to contacting at least one mobile reactant solution to at least one reactant that was previously deposited on a substrate (the deposited reactant also may have been immobilized on the substrate as described herein). This is exemplified by the block diagram of FIG. 3, which represents an embodiment of contact act 120 from FIG. 1. In act 300 of FIG. 3, a microfluidic conduit is formed by contacting the open side of a microfluidic channel to a reaction surface in an orientation such that the channel intersects at least one area (e.g., a line or a spot) of reactant previously deposited on the surface. The resulting microfluidic conduit may intersect multiple areas (e.g., lines or spots) of deposited reactant. In act 310, a volume of a mobile reactant solution is flowed into and/or through the microfluidic conduit to contact the deposited reactant at the intersection between the microfluidic channel and the area of deposited reactant. In act 320, the microfluidic channel conduit is removed from the reaction substrate in order to process the substrate for analysis in act 130. Act 320 is optional, as are other acts. Other aspects of the contact procedure may include fewer acts, additional acts, or alternative acts as described herein. It should be appreciated that an array of open microfluidic channels may be used to contact a plurality of mobile reactant solutions to one or more previously deposited reactants on a substrate. In some embodiments, the reaction between mobile reactants and surface bound reactants can be monitored directly without removing the second microfluidic channel (or array of microfluidic channels) from the reaction surface.

As is to be appreciated, embodiments of the invention may include the above described method of forming a contact 62 between one or more mobile reactant(s) and one or more immobilized reactant(s) without also completing other described methods (e.g., without using the deposition procedure described herein. In this regard, a reactant such as a component of a sample or reagent may be provided to a reactant that was previously immobilized on a microarray. Such microarrays may be produced through methods other than those previously described herein (e.g., using other reactant deposition methods known to one of skill in the art), or may be procured with one or more sample or reagent components already deposited and/or immobilized thereon.

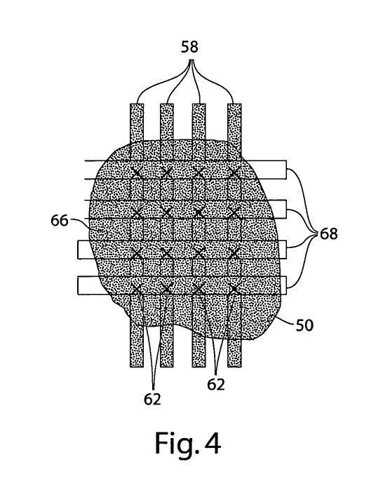

In some embodiments, a plurality of reactants may be deposited through a plurality of microfluidic channels (preferably using an array of microfluidic channels 64) onto a reaction surface to form a plurality of reactant lines as illustrated in FIG. 4. The reactant lines 58 shown in FIG. 4 are substantially parallel lines. However, the reactant lines may be configured in any way that allows subsequent analytical steps to be performed, as the invention is not limited in this respect. In some illustrative embodiments, the reactant lines may not intersect each other over an “analytical portion” 66 of the reaction substrate (the portion of the reaction substrate that is monitored for reactions between sample and reagent).

It should be appreciated that the reactant lines described herein (e.g., deposition or contact lines) may be of any shape, including linear, curved, branched, or a combination thereof. Each line may include one or more bends (e.g., curves or angles). The shape and configuration of the lines is related to the shape and configuration of the open microfluidic channels that are contacted to the reaction surface and used to deliver the reactant(s) to the reaction site(s).

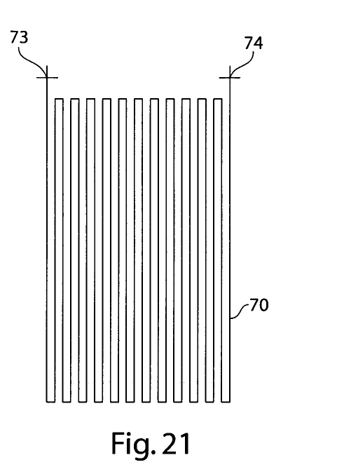

In some embodiments, a plurality of mobile reactant solutions 68 are flowed through a plurality microfluidic channels (preferably using an array of microfluidic channels) to intersect the plurality of reactant areas (e.g., lines or spots) previously deposited on the substrate. This may form a matrix of contact points 62 between the mobile reactant solutions and the immobilized reactant areas, as illustrated in FIG. 4. FIG. 4 shows an embodiment of the invention where an array of substantially parallel flows of mobile reactant solutions substantially normal to an array of substantially parallel lines of immobilized reactant. As discussed above, the different mobile reactant flows can be configured in any way that allows subsequent analytical steps to be performed. In some illustrative embodiments, the mobile reactant flows may not intersect each other over an analytical portion of the substrate surface. Additionally, in illustrative embodiments each mobile reactant flow intersects an immobilized reactant line only once. However, the invention is not limited in this regard, as the mobile reactant flow may intersect any given immobilized reactant line multiple times. In fact, in one illustrative embodiment as shown in FIG. 21, a single channel 70 may flow reactant in a serpentine manner about the analytical portion of a reaction substrate. Such an embodiment may prove particularly useful in applications where a single sample is to be distributed about a matrix of contact points on a reaction substrate. For example, a sample may be distributed over a micro-array of reactants such as an array of oligonucleotide probes that were previously deposited on the reaction surface (e.g., a commercially available micro-array). In one embodiment, one or more channels may be configured to follow a circuit that runs over a plurality of previously deposited reactant spots. This format may be suitable when the dimensions of the spots are greater than the channel width thereby allowing several channels to cross a single reactant spot on the reaction surface.

According to another embodiment, aspects of the invention provide microfluidic arrays that are useful for the reactant deposition and contact steps described above. In some embodiments, the same microfluidic array can be used for the deposition and contact steps. However, in illustrative embodiments, different channel configurations may be used for the deposition and contact steps, as the present invention is not limited in this respect.

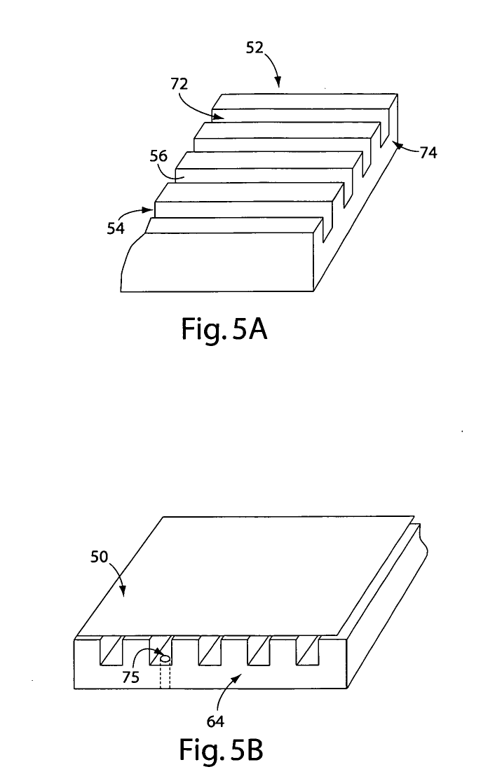

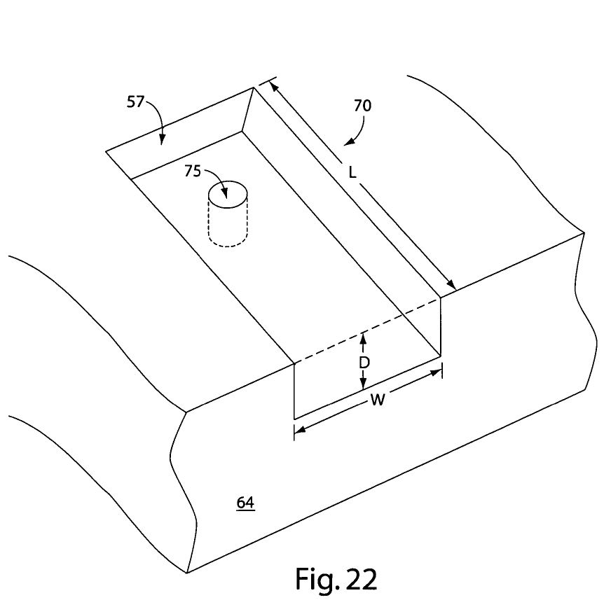

FIG. 5a shows a portion of an exemplary microfluidic array. In FIG. 5a, each channel 70 has a single channel inlet 72 toward a first end, a single channel outlet 74 at a second end, and a channel wall 56 separating each adjacent channel of the array. However, in other embodiments, each channel could have two or more channel inlets and two or more channel outlets. Still, the channels of other embodiments may share common inlets or outlets, as aspects of the present invention are not limited to any particular channel configuration. As described herein, each channel may have end(s) defined by end walls 57 (see FIG. 22, for example) not shown in the portion of the array of FIG. 5. Accordingly, the inlet and outlets may not be open cross-sections at the ends of the channels. Rather, inlets and/or outlet ports may be included in the form of one or more holes 75 connecting one or more walls (e.g., a channel side, end, floor, or combination thereof) to an opening on another side of the array (see FIGS. 5 and 22 for example).

As described in more detail herein, one or more of the channel outlets may be connected to an exhaust or evacuation channel 76. One or more of the channel outlets also may be blocked to form a dead end channel without an outlet. Still, one or more of the channel outlets also may be connected to a well 78 of a multi-well plate 80 or other reservoir, either directly or indirectly, such as through a fluidic channel of a transfer plate, as described in greater detail herein.

In another embodiment, aspects of the invention provide a reaction substrate for use with an array of microfluidic channels, or “microfluidic array” 64 as used herein. In some embodiments, this substrate is a standard glass plate. In other embodiments, the substrate has structural features that are useful to align the substrate with the microfluidic array. Similarly, the microfluidic array can have structural features that are useful to align the reaction substrate with the microfluidic array. Other structural features in either one or both of the microfluidic array and the reaction substrate can be included to form and maintain a fluid seal between the two components while solutions are flowed through the channels. In some embodiments, the reaction substrate may include sample areas (e.g. lines or spots) that were previously immobilized on its surface, either through methods described herein, or other methods.

In a further embodiment, aspects of the invention may include a transfer plate 84 to connect an array of microfluidic channels to a sample array such as a microtiter or multi-well plate or a multi-pippette delivery device. The transfer plate may adapt the microfluidic array for use with automated sample processing devices and methods that operate at a larger scale. A docking device 82 may be used to interface a sample delivery device with the transfer plate.

The invention also provides additional configurations of docking devices, transfer plates, and microfluidic channels that can be used to deposit or react a single reactant or a plurality of reactants on a substrate surface (e.g., to form a matrix of reactants).

As used herein, reactants may be components of a sample to be assayed (e.g., a biological sample such as a tissue extract, blood serum, urine, sputum, extracted cell protein, microorganisms, an environmental sample, a sample to be tested for a biologically active or infectious organism, a sample to be tested for a chemical moiety, a sample to be tested for a toxin or other harmful molecule). Accordingly, a reactant may be a nucleic acid (e.g., genomic DNA, other DNA, or RNA), protein, polypeptide, lipid, carbohydrate, other metabolite, or combination thereof. A reactant also may be any other moiety that can be either deposited on a reaction surface and/or flowed across a reaction surface in the form a reactant solution and which may be involved in a reaction with another reactant. A sample may be obtained from an animal, plant, microbe, or virus. An animal may be, for example, a mammal (e.g., a human, mouse, rat, dog, cat, horse, cow, goat, sheep, primate, etc.), a bird, or a reptile. A biological sample solution may be a crude, partially purified, or substantially purified solution containing one or more reactants as described herein. A reactant may be purified according to procedures known to one of skill in the art. Reactants also may be components of a reagent used to detect or otherwise perform an assay on a sample. Accordingly, a reactant may be a nucleic acid probe (e.g., a DNA, RNA, PNA, or modified form thereof), a peptide, an antibody, an aptamer, a binding agent, an enzyme substrate. A detection reactant may be labeled (e.g., with a fluorescent, enzymatic, radioactive, magnetic, electromagnetic, or other detectable label, or a combination thereof). A reagent solution may contain one or more different reactants. It should be appreciated that sample and reagent solutions also may include buffers, salts, and other components (e.g., blocking agents, nucleotides, other metabolites, enzymes, etc.). A reagent solution may contain components suitable for an enzyme reaction, including buffer and substrates for the enzyme. A reagent solution may contain components suitable for a nucleic acid amplification reaction (e.g., PCR, LCR, rolling circle amplification or other isothermal amplification, etc.) that may be useful to promote or stabilize desirable reactions. It should also be appreciated that aspects of the invention may be practiced by depositing either one or more sample reactants on a surface or by depositing one or more detection reactants on a surface, as the invention is not limited by the type of reactant that is deposited on a reaction surface. Similarly, either a mobile sample reactant or a mobile detection reactant may be contacted to a previously deposited reactant, as the invention is not limited by the type of mobile sample reactant. In some embodiments, a combination of sample and detection reactants may deposited on a reaction surface (either mixed together or separately). In some embodiments, a combination of mobile sample reactants and mobile detection reactants may be used (either mixed together or separately).

These and other aspects of the invention are described in more detail herein.

Microfluidic Methods for Depositing a Reactant on a Reaction Surface

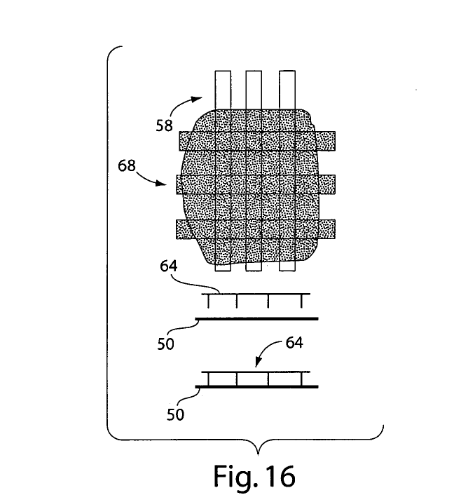

As outlined in FIG. 2, a reactant (e.g., a sample or reagent) may be deposited on a reaction surface of a reaction substrate using an open microfluidic channel. The open side of the microfluidic channel is covered with a reaction substrate to form a closed channel or conduit. FIGS. 5B and 16 show a reaction surface 50 in contact with the open side of a portion of a microfluidic array 64. The reaction substrate is shown in direct contact with the top surface of the side walls of the open microfluidic channel, thereby forming a closed channel with part of the reaction surface forming a wall of the closed channel. As discussed herein, the closed channel may have end walls shown in FIG. 22 and channel inlets and/or outlets are provided by inlet or outlet ports. A solution of the reactant to be deposited onto the surface is then flowed into the channel. This may occur with the reaction substrate positioned beneath the microfluidic array as shown in FIG. 16, or above as shown in FIG. 5b, or in other orientations. The orientation of the reaction substrate and associated microfluidic channels is not limiting. The substrate may be above the channel array, the channel array may be above the substrate, the substrate may be on its side or end with a channel array next to it, the substrate and associated channel array may be rotated in any direction that is convenient for the operator and/or device being used. However, it may be important for the seal formed between the walls of the channels and the reaction surface to be adapted to be sufficiently leak-proof for the orientation being used. The efficiency of reactant deposition is a function of several factors, including the concentration of reactant in the solution, the speed of reactant solution flow in the channel, the time of contact between the solution and the reaction surface, the volume of reactant solution that is flowed through the channel, and the physical properties of the reaction surface.

In one embodiment, the channel may be filled with sample solution and incubated for a time sufficient for sample deposition. The time required for deposition depends on several factors as discussed herein. However, times range from nearly instantaneous to several hours. The sample solution can be removed by flushing with another solution, and/or be dried by flushing with a gas such as air or nitrogen. Still, in other embodiments the sample may be of a small enough volume that drying occurs almost immediately. The sample solution could also be removed by removing the microfluidic channel. However, if the channel is full, reaction solution may spill over the reaction surface and blur the line of reactant deposited by the microfluidic channel.

In another embodiment, a volume of sample may be flowed through a channel. The volume may be sufficient to fill the height and width of the channel as the volume is flowed through the channel. The volume should be sufficient to ensure contact between the reaction surface and the solution as it flows through the channel. As the volume progresses through the channel, reactant may be deposited on the reaction surface. After the volume has flowed through the channel, a trail or line of reactant remains on the reaction surface. In some embodiments, the sample volume is surrounded by air or other gas as it flows through the channel. As a result, once the volume has flowed through the channel, the deposited sample line is relatively dry and will not mix with any adjacent sample lines when the microfluidic channel is removed from the reaction surface. Accordingly, in one embodiment, one or more reactants may be deposited using an uninterrupted flow of a solution volume through the channel and across the surface (i.e., the flow of the solution is not stopped at any time during the deposition). Aspects of the invention are not limited by the flow speed of the reactant solution. However, a slower flow speed may result in increased deposition efficiency of reactant provided that the flow speed is not so slow that the solution is essentially depleted of reactant (or reactant diffusion to the surface is rate limiting) at a position in the channel before fresh solution is introduced to that position.

In one embodiment, a volume of reactant solution that is larger than the volume of the portion of the closed channel formed by contact with the reaction surface (i.e. smaller than the volume of the portion of the channel that is in direct fluid contact with the reaction surface) may be flowed through the channel. The volume may be drawn (by vacuum) or pushed (by positive pressure) from a reservoir upstream from the reaction area, as described herein. In aspects of the invention, the volume of reactant solution that is flowed through a channel may be 10 to 1,000 times greater than the volume of the portion of the channel that is in contact with the reaction surface. However, aspects of the invention are not limited by the reactant volume. Accordingly, in one embodiment, a reactant volume may be smaller than the volume of the portion of the closed channel formed by contact with the reaction surface (i.e. smaller than the volume of the portion of the channel that is in direct fluid contact with the reaction surface).

In one illustrative embodiment, the channel is 50 microns wide (dimension “W”), 10 microns deep (dimension “D”), and the contact length with the reaction surface is 10 mm long (dimension “L”), as illustrated in FIG. 22. The contact length with the reaction surface may be the length between the two end walls of an open channel. Alternatively, the contact length may be the length of open channel if the channel(s) include one or more closed portion(s). In one embodiment, the analytical length (the contact length with the analytical portion of the reaction surface) may be shorter than the contact length with the reaction surface. In particular, different configurations of channels may include one or more portions of the channel that are for delivering a solution from an input port to the analytical portion of the reaction surface, or for exhausting a reactant solution to an outlet port. As described herein, due to certain geometrical constraints imposed by having large numbers of channels, the analytical portion of the array may be located remotely form the inlet and outlet ports. The analytical length of a channel may be between 1 mm and 5 cms, and preferably about 1 cm long. However, any analytical length may be used. Longer analytical lengths may be required to interrogate more immobilized reactants (e.g., more deposited lines of reactant. In one embodiment, a the analytical length or portion of a channel may be narrower that either one or both of the upstream (delivery) or downstream (exhaust) portions of the channel. For example, the width of the delivery and exhaust portions of the channel may be between about 100 and 200 microns, whereas the width of the analytical portion of the channel may be between about 10 and about 90 microns. However, other combinations of sizes described herein may be used. Similarly, in some embodiments, the channel depth in the analytical portion of the channel may be smaller than that of the delivery and exhaust portions.

In one aspect, a nucleic acid sample can be deposited by flowing a 100 nanoliter volume of nucleic acid solution across the 10 mm contact length in 60 seconds. The nucleic acid sample may contain between 1 and 100 nanograms of DNA, more preferably about 10 nanograms of DNA. An oligonucleotide (e.g., a labeled oligonucleotide) concentration may be between 1 and 1,000 nM. The nucleic acid sample volume can be between 1 and 100 nanoliters. In some embodiments, a smaller or larger volume may be used. In some embodiments, between 100 nanoliters and 5 microliters of reactant solution may be flowed through a channel. In one embodiment, 500 nanoliters of reactant solution may be flowed through a channel. A flow time may be between about 1 and about 5 minutes. However, shorter or longer flow times, and smaller or larger volumes may be used. It should be appreciated that shorter flow times may be used with solutions having higher reactant concentrations.

Sample solution flow may be induced, in one illustrative embodiment, by applying a vacuum to one portion of channels of the microfluidic array. For example, a vacuum line can be connected to the channel either through the reaction substrate or the substrate comprising the microfluidic array, or both. However, the vacuum may be applied elsewhere, or not at all, as the invention is not limited in this respect.

The microfluidic array may be removed from the reaction surface to allow for additional processing steps. The reaction surfaces can be washed, dried, and treated in additional ways to immobilize the sample that was deposited on the reaction surface. Examples of methods for strengthening the interaction between a sample and a reaction surface are known in the art and depend on the nature of the sample and the reaction surface. For example, nucleic acids can be fixed onto a glass surface by treatment with ultraviolet light, heat, or both. However, as is to be appreciated, embodiments of the invention may not require any additional steps, as the invention is not limited in this manner.

Embodiments of the invention may produce an essentially continuous line or lines of sample on the reaction surface. The shape and size of the line may be a function of the shape and size of the microfluidic channel that was used to deposit the sample. A single channel may have sections of different shape and size (e.g., different width and/or height).

In one illustrative embodiment, multiple samples are deposited simultaneously. In one embodiment, different samples are deposited in parallel lines using an array of microfluidic channels. The channels are preferably connected to one or more exhaust channels or ports that collect the samples after they flow across the reaction surface. The exhaust ports are typically connected to one or more waste containers. However, sample solutions could be retrieved in individual containers for subsequent use.

In some embodiments, a sample volume is flowed back and forth across the reaction surface in order to deposit the appropriate amount of sample on the surface.

The amount of sample to be deposited depends on the nature of the sample and the assay that will be performed on the sample.

Microfluidic Methods for Contacting an Immobilized Reactant with a Mobile Reactant Solution.

Reactants that are deposited (and/or immobilized) on a reaction surface of a reaction substrate can be contacted by one or more mobile reactants using a microfluidic channel. According to the invention, the area of the deposited reactant is determined by the method used to deposit the reactant. One or more reactants may be deposited using methods of the invention or other deposition methods (including spotting and lithography as described herein, electrochemical deposition (e.g., as described in Egeland et al., 2002, Anal. Chem., 74, 1590-1596), and/or other techniques known to one of skill in the art).

It should be appreciated that methods and devices described above and below for depositing a reactant on a reaction surface also may be used for contacting an immobilized reactant with a mobile reactant solution. Similarly, methods and devices described above and below for contacting an immobilized reactant with a mobile reactant solution also may be used for depositing a reactant on a reaction surface.

In one embodiment, a microfluidic channel may be contacted to a reaction surface in such a way that the channel intersects one or more areas of immobilized reactant on the surface. A solution of the mobile reactant then may be flowed into the channel. In some illustrative embodiments, the reactants may be continuously moved over the immobilized reactant—never remaining stationary. The time required for the interaction between mobile and deposited reactants depends on the nature of the interaction and the concentrations and volumes of the reactants. In an embodiment of a nucleic acid hybridization reaction, no stationary hybridization time may be used, because detectable and representative hybridization occurs within the time that it takes for the volume of mobile reactant solution to pass over the immobilized reactant. Similarly, in embodiments of other reactions (e.g., antibody/antigen interactions) no stationary reaction time may be used. However, in some embodiments, the mobile reactant solution may be left in the channel for a sufficient time to interact with the immobilized reactant. The mobile reactant can be left for between about 5 seconds and 12 hours. In other embodiments it can be left for shorter or longer times. In some embodiments, the mobile reactant is left for 1 to 6 hours, or 6 to 12 hours. The optimal reaction time may be dependent on the concentration of the mobile reactant in solution. In some embodiments, 60 picomoles of Cy3 and Cy5 labeled probes were prepared in a solution volume of 0.5 microliters. It should be appreciated that if a mobile reactant solution is to be left stationary over the deposited reaction area for a reaction time, it may be desirable to use a sufficient volume of mobile reactant solution to cover the deposited reactant area during the reaction or interaction time. In contrast, if no reaction or interaction time is being used, the volume of mobile reactant can be such that it contacts the entire deposited reactant area by flowing over it, but it may not cover the entire area at any single point in time. In some embodiments, a small volume of the mobile reactant could be flowed over the immobilized reactant as described for the deposited reactant above. However, in other embodiments, the volume may be large enough to cover substantially all of the reaction surface during the flow time (except for during channel filling and emptying).

The reaction surface can be treated before the mobile reactant is contacted to the surface to prevent any interaction between the reactant and the surface.

In one illustrative embodiment, a plurality of mobile reactants are contacted to one or more immobilized reactants on the reaction surface using an array of microfluidic channels. Typically, the immobilized reactants are deposited in parallel lines on the surface and the mobile reactants are flowed across the immobilized reactant lines so that every mobile reactant flow intersects every sample line. However, in some embodiments, a subset of sample lines and mobile reactant lines may not intersect. Typically, the mobile flow lines are perpendicular to the immobilized sample lines. However, any angle (or combination of angles) between different sets of lines can be used provided the desired number of immobilized reactants are contacted with mobile reactant lines.

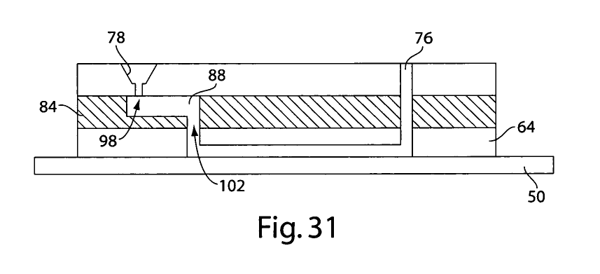

According to aspects of the invention, each channel can be connected to an evacuation channel or port 76 as discussed herein. Accordingly, in some embodiments, there may be a single evacuation port in the form of a through-hole for each channel. In this embodiment, the evacuation port is the channel outlet port. In other embodiments, the outlets of different channels may merge to form one or more common evacuation channels that may be connected to an evacuation port that may be in the form of a through-hole as described herein. In one embodiment, several channels (e.g. about 2, 3, 4, 5, 10, 100, or more) may be connected to a single evacuation port or channel. Shared outlets may be connected in the form of a tree or manifold with shared evacuation ports or channels from a few microchannels merging into larger common evacuation ports or channels. There may be several stages of channel merging leading to one or a few common evacuation ports or channels for the entire array of microfluidic channels. At each stage, a common channel formed by the merging of several other evacuation channels may have a larger cross-sectional area (e.g., wider, deeper, or both) than each of the channels that merged. In one embodiment, the cross-sectional area of a larger common channel may be identical to the sum of the cross-sectional areas of each of the smaller channels that were merged to form the larger channel. In one embodiment, the cross-sectional area of a common channel formed by the merging of several smaller channels may be identical or substantially identical to the cross-sectional area of each of the channels that were merged. According to aspects of the invention, even when the outlets are merged, each channel may still have a single inlet connected to a single reactant loading port (optionally through a transfer plate). In one embodiment, a reactant solution may be drawn into an array of microfluidic channels by applying a vacuum to one or more of the exhaust channels or ports. The vacuum pressure may be equal on all exhaust channels or ports. In one embodiment, about 1.5 psi of vacuum may be applied. However, any suitable positive or negative pressure may be applied. As described herein, an evacuation port 76 may pass through several devices including an array, a transfer plate, a reservoir plate, and/or a docking interface (see FIG. 31, for example).

In some embodiments, one or more of the channels may be dead end channels. Reactant solutions may readily be forced into dead end channels if the microfluidic array material is sufficiently porous to allow diffusion of any gas trapped by the advancing reactant solution. In one embodiment, a dead end channel may contain a mobile reactant in the channel and may allow for prolonged contact between the modile reactant and the immobilized reactant(s) on the reaction surface. When using a dead end channel, the flow of the mobile reactant(s) may not need to be monitored. Indeed, the mobile reactant(s) may be introduced into the channels and driven to the dead-ends using 1-3 psi of pressure (or other appropriate amount of positive or negative pressure) without requiring sophisticated monitoring equipment to ensure that a sufficient amount of reactant is in each microfluidic channel. In one such embodiment, the channels are dead-ended within the area of contact between the reaction surface and the microfluidic array.

In one embodiment, after sufficient incubation, the channel or array of channels may be removed and the reaction surface may be washed to remove any unbound reactants prior to further analysis. This washing step may be used where a) the reaction product to be detected remains associated with the immobilized surface sample, and b) mixing of reagents upon removal of the array does not interfere with the detection and interpretation of the reaction results. However, as is to be appreciated, the invention is not limited by requiring washing steps.

In some instances, such as assays involving hybridization of nucleic acids, it may be desirable to control the temperature of the reaction surface and/or microfluidic channel. This may be achieved using a variety of conventional means. For example, if either component contains an appropriate conductor, such as anodized aluminum, that component may be contacted with an appropriately controlled external heat source. Alternatively, the channels could be outfitted with heaters and thermocouples to control the temperature of the fluid disposed within them or running through them.

The method by which an interaction between an immobilized and a mobile reactant is analyzed will depend upon the reactants. For example, where the two chemical species each constitute one member of a binding pair of molecules (for example, a ligand and its receptor or two complementary polynucleotides), the interaction can be conveniently analyzed by labeling one member of the pair, typically the chemical species in solution, with a moiety that produces a detectable signal upon binding. Only those contact points where binding has taken place will produce a detectable signal.

Any label capable of producing a detectable signal may be used in embodiments of the invention. Such labels include, but are not limited to, radioisotopes, chromophores, fluorophores, lumophores, chemiluminescent moieties, etc. A label may be a compound capable of producing a detectable signal, such as an enzyme capable of catalyzing, e.g., a light-emitting reaction or a calorimetric reaction. A label may be a moiety capable of absorbing or emitting light, such as a chromophore or a fluorophore.

Alternatively, both chemical species may be unlabeled and their interaction may be indirectly analyzed with a reporter moiety that specifically detects the interaction. For example, binding between an immobilized antigen and a first antibody (or vice versa) could be analyzed with a labeled second antibody specific for the antigen-first antibody complex. For nucleic acids, the presence of hybrids could be detected by intercalating dyes, such as ethidium bromide, which are specific for double stranded nucleic acids. In another embodiment, an interaction between unlabeled reagents may be detected using plasmon resonance imaging. In one aspect, a technique for detecting an interaction between two or more reactants may involve flowing two or more mobile reactant solutions sequentially over an immobilized reactant.

Once patterned in the horizontal and vertical directions, the glass slide is free to be analyzed using any detection device including, but not limited to, a standard slide scanner.

Those of skill in the art will recognize that the above-described modes of detecting an interaction between two reactants at a contact point are merely illustrative. Other methods of detecting myriad types of interactions between chemical species are well known in the art and can be readily used or adapted for use with the arrays of the present invention.

It should also be appreciated that methods and devices described for depositing a reactant also may be used for delivering a mobile reactant in some embodiments. Similarly, methods and devices for delivering a mobile reactant may be used for depositing a reactant in some embodiments. It should also be appreciated that methods and devices described with merged and/or common outlet channels may be flowed in the opposite direction to deliver a common reactant to a plurality of channels. In such applications, an array may have branching channels both upstream and downstream form the analytical portion of the channels.

Reaction Substrates

Useful reaction substrates may have a reaction surface with properties that do not interfere with reactant (e.g. sample or reagent) deposition in step 110. For example, if the sample is negatively charged, a negatively charged reaction surface may be avoided for some embodiments. Similarly, a reaction surface may be one that does not interfere with subsequent reaction and detection steps 120 and 130. For example, it may be desirable for a reaction between a reactant bound to a reaction surface and a mobile reactant to not be obscured by a reaction between the mobile reactant and the reaction surface. For example, in a DNA hybridization reaction where target DNA is immobilized on a reaction surface, the hybridization of a mobile labeled probe to its complementary target sequence should be stronger than the binding of that probe to the reaction surface, at least according to some embodiments.

A reaction surface may be a flat or substantially flat surface. Alternatively, a reaction surface may include a regular or irregular pattern of bumps, stipples, ripples, valleys, hills, mounds, one or more mesh-like structures, or other physical variations. Depending on the intended use, a reaction surface may be porous, hydrophilic, hydrophobic, negatively charged, positively charged, sticky, or a combination thereof. Regions of the reaction surface may have different properties, and a reaction surface may include one or more areas with any one or more of the properties described herein. However, in many embodiments the reaction surface (or a portion thereof) is such that it can form a leak-proof (or substantially leak-proof) seal when contacted by the walls of one or more microfluidic channels.

A reaction substrate may be a single layer of material having a reaction surface. Alternatively, a reaction substrate may include two or more layers where a reaction surface layer is supported by one or more underlying support layers. Different layers may consist of different material. A reaction surface may be treated (e.g., physically or chemically) before a reactant is deposited onto the surface. The treatment may be suitable for improving the binding or other properties of the reaction surface as described herein.

In some embodiments, a reaction surface may be treated (e.g., physically or chemically) after a first set of samples is deposited in order to prevent or minimize any interaction between the reaction surface and a second set of samples. For example, after target DNA is deposited (and preferably immobilized) on a glass surface for a hybridization assay, the glass surface may be treated with a blocking agent such as salmon sperm DNA to prevent non-specific binding between the glass surface and any subsequently added hybridization probes.

In many embodiments of the invention, the deposited sample is a biological sample. The reaction surface may be sensitized to bind to a reactant that is to be deposited on the surface. For example, the reaction surface may be modified by attachment with or otherwise coating with a biomolecular recognition species. Useful biomolecular recognition species include a protein (e.g., an antibody, an antibiotic, an antigen target for an antibody analyte, or a cell receptor protein), a nucleic acid (e.g., DNA or RNA), a cell, or a cell fragment.

If a biomolecular recognition species is to be added to the reaction surface, the surface may be composed of a material or mixture of materials that may be readily activated or derivatized with reactive groups suitable for effecting covalent attachment. Non-limiting examples of suitable materials include acrylic, styrene-methyl methacrylate copolymers, ethylene/acrylic acid, acrylonitrile-butadiene-styrene (ABS), ABS/polycarbonate, ABS/polysulfone, ABS/polyvinyl chloride, ethylene propylene, ethylene vinyl acetate (EVA), nitrocellulose, nylons (including nylon 6, nylon 6/6, nylon 6/6-6, nylon 6/9, nylon 6/10, nylon 6/12, nylon 11 and nylon 12), polycarylonitrile (PAN), polyacrylate, polycarbonate, polybutylene terephthalate (PBT), polyethylene terephthalate (PET), polyethylene (including low density, linear low density, high density, cross-linked and ultra-high molecular weight grades), polypropylene homopolymer, polypropylene copolymers, polystyrene (including general purpose and high impact grades), polytetrafluoroethylene (PTFE), fluorinated ethylene-propylene (FEP), ethylene-tetrafluoroethylene (ETFE), perfluoroalkoxyethylene (PFA), polyvinyl fluoride (PVA), polyvinylidene fluoride (PVDF), polychlorotrifluoroethylene (PCTFE), polyethylene-chlorotrifluoroethylene (ECTFE), polyvinyl alcohol (PVA), silicon styrene-acrylonitrile (SAN), styrene maleic anhydride (SMA), metal oxides, and glass. In some embodiments, the reaction surface also could be composed of PDMS or polymethyl methacrylate (PMMA) or any combination of suitable materials, including those described herein.

In particular embodiments, antibodies can be immobilized on the reaction surface using methods known in the art. In other embodiments, ligands and or antigens can be immobilized on the reaction surface.

The size and shape of the reaction surface may depend on several factors, including the number of reactions to be performed, the size of the sample channels, and the size of the array of channels. However, since aspects of the invention bring mobile reactant solutions to immobilized reactants by active fluid flow, the efficiency of each reaction is not affected by the size of the reaction surface. Therefore, the reaction surface may be significantly larger than many currently used microarrays. Accordingly, the surface can be sized to accommodate as many reactions as needed. This may be an advantage over current microarray systems that are limited in size, because they rely on diffusion between reagents and bound targets over the entire surface of the array as opposed to diffusion only over the size of the reaction contact. Nonetheless, in one embodiment, substrate sizes may be similar to those of other microarray systems so that the substrates can be processed using available automated devices and procedures. Similarly, aspects of the invention are not limited by the shape of the reaction substrate and reaction surface. They may be subtantially rectangular, square, circular, oval, or other regular or irregular shape. Aspects of the invention also are not limited by the thickness of the reaction substrate. However, in some embodiments, the reaction substrate may be between about 0.1 mm and 10 mm. The thickness of the reaction substrate may be related to the physical properties (e.g., the strength and/or flexibility) of the substrate material.

As discussed above, a reaction surface may be flat so that it readily forms a seal with the upper surface of the microfluidic channel walls upon contact. However, other shapes also may be used as the invention is not limited in this regard.

Microfluidic Arrays

According to aspects of the invention, microfluidic arrays 64 may reduce the cost of, increase the speed of, and/or increase the accuracy of many assays including hybridization tests in various applications. In one aspect, one or more microfluidic channel(s) may be used to run a volume of reactant solution over a reaction surface. In one embodiment, a reactant may be deposited on the reaction surface. In an alternative embodiment, a reactant may be brought into contact with another reactant that was previously deposited on the surface as described herein.

Aspects of the invention provide a novel platform that enables a large number of individual data points to be obtained by interrogating a group of samples with a group of reactants such as probes. Microfluidic channels, preferably arranged as a microfluidic array, may be used to contact columns of mobile reactant to rows of deposited or immobilized reactants as discussed herein.

In some embodiments, each row of sample may interact with all of the columns of probes, thus providing a novel assay platform where each intersection of sample and probe represents a unique data point. The number of samples or the number of probes used may be varied from one to the largest number that a column or row of the device will hold. In one embodiment, a microfluidic array may be used with a standard 25 mm by 75 mm microarray glass slide to obtain 1536 lines of sample running in the short direction, and three times 1536 lines of probe (4608 probes) running in the long direction. Multiplexing of these two groups results in each of the 1536 sample being probed for 4608 targets, totaling over 7 million unique data points. In contrast, when using currently available microarrays, only one to two samples are typically tested against probes, although the probe number is generally in the tens of thousands. For example, the Affymetrix HUSNP chip interrogates 10,000 targets, but only in a single sample.

According to the invention, microfluidic conduits useful for exposing a solution to a reaction surface are formed by contacting the open side of an open microfluidic array of channels to the reaction surface, thereby forming a closed microfluidic channel or conduit along the length of the contact. An open microfluidic channel of the invention comprises a channel floor and a pair of guiding walls that are typically used to direct the sample when it is flowed across the reaction surface and therefore to determine where samples are deposited on the surface. A representative array of channels, as shown in FIGS. 5a and 5b, is formed in a substrate having a plurality of channel walls. The walls may be parallel and may separate parallel microfluidic channels, although the invention is not limited in this regard. The thickness of each wall is typically similar to the width of each microfluidic channel. However, different wall thicknesses and different channel widths can be used. Wall thicknesses may range from about 1 micron to about 200 microns, and may be between about 5 microns and about 150 microns, and may be about 100 microns. However, smaller or larger wall thicknesses may be used. Similarly, channel widths and heights range from about 1 micron to about 500 microns, and may be between about 5 microns and about 250 microns. In some embodiments, channel widths and/or heights may be between about 1 micron, about 10 microns, about 20 microns, about 30 microns, about 40 microns, about 50 microns, about 100 microns, about 150, about 200 microns, or about 250 microns. However, other sizes including smaller or larger sizes also may be used as aspects of the invention are not limited in this regard. The height and width of a channel may be independent. However, in one embodiment, the height and width of a channel may be substantially the same in order to optimize pressure gradients and or fluid flow patterns. However, smaller or larger channel widths and heights can be used. Channel lengths are usually similar to the linear dimensions of a standard glass slide. In some embodiments, channel lengths may range form about 5 mm to about 5 cm. However, any channel length can be used provided that the length does not prevent solution flow in the channel. Channels may be of uniform length, width, and/or depth. However, aspects of the invention are not limited by the size and configuration of the channels. Accordingly, a microfluidic array may include one or more channels and each channel may have a different length, height, width, and/or configuration. In one embodiment, a channel may

Each channel may have ends defined by the ends of the open portion of the channel on the channel surface of the array. As used herein, the channel surface of the array is the surface that presents one or more open channels. Each channel may not extend to the edges of the array. Accordingly, when a reaction surface is contacted to one or more open channels, a closed system is formed provided that the reaction surface covers the entire open portion of each channel. Accordingly, in order to deliver a solution to a closed conduit, each microfluidic channel may be in fluid communication with an inlet port. The inlet port maybe a through hole that connects the channel surface of the array (e.g. the channel wall, floor, or combination thereof) to another surface of the array (e.g., the surface of the array that is opposite to channel surface). This inlet port can be used to load a solution directly into the channel, e.g. using a microfluidic loading device inserted into the port. In one embodiment, the microfluidic loading device may be an interface or docking device of the invention. Alternatively, the inlet port can be connected to a transfer plate as described herein. Similarly, each channel may be in fluid communication with an outlet port. An outlet port also may be a through hole connecting the channel surface of the array to another surface of the array. In one embodiment, the outlet port may be connected to a vacuum, either directly or via a transfer plate as described herein. The outlet port may be used to remove solution from the channel and may be in fluid communication with one or more channels or outlet holes on a transfer plate as described herein. The inlet and outlet ports may be located approximately at the ends of each channel on the microarray. The location of an inlet and/or outlet port is not limiting, and a port may be located anywhere along the length of a channel.

An array of microfluidic channels can be mated with a reaction substrate such that the open side of each channel is facing downward toward the substrate. When a reactant solution is flowed through each channel in the array, it may be guided by the sidewalls, and potentially, the top wall of each channel so that the sample may be deposited onto the reaction surface as described herein.

Microfluidic arrays may contain any number of microfluidic channels. In some embodiments, a microfluidic array has between 5 and 500,000 microfluidic channels. However, smaller or larger numbers of microfluidic channels can be supported by a microfluidic array. Some illustrative embodiments of microfluidic arrays have between about 10 and 100,000 microfluidic channels, preferably between 100 and 10,000, more preferably around 1,000 or 2,000, and up to about 5,000. Still, other embodiments of microfluidic arrays may comprise a single microfluidic channel, such as the one defining a serpentine path in FIG. 21. In one embodiment, the number of channels per linear centimeter (measured on the array in a direction that crosses a plurality of channels) may be between about 10 and about 500, or between about 50 and about 250. In one embodiment, the number of channels per linear centimeter may be about 40, about 60, about 80 about 100, about 120, about 140, about 160, about 180, about 200, about 300, about 400, about 500. However, other numbers, including larger or smaller numbers may be used. It should be appreciated that the number of channels on the array may determine the number of reactants that may be deposited and/or reacted according to aspects of the invention. Accordingly, the number of interaction sites can be calculated by multiplying the number of deposited reactant lines by the number of mobile reactant lines that are used in some aspects of the invention.

Microfluidic arrays are preferably made of a material, such as the materials typically used in soft lithography. As is to be appreciated, such materials may be soft enough to provide a seal when mating with the substrate. However, a separate sealing gasket may be used to prevent leakage of the sample fluid between the channels or out of the entire microfluidic array. This may be implemented when the microfluidic array is made of a harder material, such as silicon, that may not readily seal with a substrate that is also made of a hard material.

An array is typically made by painting (e.g. spraying or spin-coating) a photoresist onto a surface such as a glass or silicon surface (e.g. a silicon wafer), exposing the photoresist to light to cure a predetermined pattern in the shape of the desired array. The uncured photoresist is removed thereby generating a mold that is subsequently used to make the array. The array is preferably made out of PDMS or polyurethane. Other possible materials include PDMS and hard plastics such as Polycarbonate or Acrylic. A PDMS device may be too costly or may absorb an unacceptable amount of biological material. A hard plastic device presents challenges in sealing the layers to each other and to the glass. However, other manufacturing methods can also be used.

In some embodiments, different structures may be used to guide the sample along the substrate surface. For instance, the mirofluidic channels are not required to have a rectangular or square cross section, nor are they required to follow linear paths as the invention is not limited in this respect. In some embodiments, channels may have a triangular cross section. Such triangular channels allow a greater percentage of the channel cross sectional area to be in direct contact with the mating substrate, which may be advantageous for depositing some samples. In other embodiments, the channel cross section reduces the percentage of contact area between channel and the mating substrate.

The reactant fluid alone may flow through each channel of an array allowing reactant to be deposited on a substrate surface. In aspects of the invention, reactant may diffuse from a reactant solution to an area of reactant surface immediately adjacent to a column of the reactant solution (e.g., below or above depending on the orientation of the reaction surface and associated microfluidic channel). In some embodiments, a reactant surface may attract a reactant. This may be the case with a glass substrate, which generally attracts charged DNA molecules. In some embodiments, it is preferable to have the channels made of an inert material, such as PDMS, polyurethane, or other perfluorinated elastomer. In this manner, the channel material may not deplete the reactant solution and may not reduce the concentration of reactant (e.g. DNA) being deposited on the reaction surface.

As discussed above, different reactants can be deposited on the reaction surface as different reactant solutions flow through the channels of a microfluidic array. Alternatively, reactant flow through each channel can be halted to allow the reactant to attach to the substrate.

As discussed herein, in some embodiments, a channel may be only partially filled with a reactant solution and as the solution passes through the channel, it may contact the reaction surface along the length of the exposed channel(s) in the microarray. In some embodiments, reactant volumes for use in a deposition (or reaction) step may be between 1/10 and 9/10 of the volume of the open portion of the microfluidic channel that contacts the reaction surface. However, smaller or larger volumes also may be used, as aspects of the invention are not limited in this respect. In some embodiments, the reactant volume may be about ½ of the volume of the microfluidic channel discussed above.

The reactant fluid may be flowed through the microfluidic array through a variety of ways. In one illustrative embodiment, the fluid is drawn though the microfluidic array by a vacuum applied at one end of each channel. However, the invention is not limited in this respect, as positive pressure may also be applied at the opposite end of each channel to drive the fluid. Still, in other embodiments, natural gravity forces associated with the sample may drive the fluid through the channels. This natural gravity force can be augmented, or replaced by a centrifugal of a centrifuge or other similar device to urge the sample fluid through the channels of the device. Additionally, other body types of forces, such as electrical forces may be used to drive the fluid through the device, such as those typically involved in electrophoretic or electrosmotic devices. In one embodiment, surface tension may be sufficient to draw a reactant solution into a channel or conduit. For example, the walls (and/or floor) of the channel, the reaction surface, or a combination thereof may be sufficiently wettable (e.g., hydrophilic) to draw an aqueous solution into a conduit.

Multi-Component Assemblies

Many microfluidic devices have proposed schemes for reducing the size and therefore improving the efficiency of existing assay procedures. However, the challenge of delivering samples and reagents to miniaturized assay devices remains a problem for many such apparatuses. Often, the added inefficiencies associated with delivering samples or reagents to miniature assay devices outweigh any efficiency gains associated with the devices. To address such problems, a transfer plate may be used to deliver sample and/or reagent to a microfluidic array from a standard laboratory device such as a multi-well plate or other macro-scale reservoir.

Multi-well plates are commonly used in industry and many automated devices and methods have been developed to streamline their manipulation in performing assays. Such automated devices may readily deliver sample and/or reagent to any standard multi-well configuration (e.g., 96, 192, 384, 768, or 1536 well configurations) using existing automation equipment. A transfer plate, according to some embodiments of the invention, may then be used to enable sample and/or reagent solutions deposited in a multi-well plate to be transferred efficiently to a microfluidic array and onto or across a reaction substrate where hybridization or other assays may be conducted.

Standard multi-well plates (also referred to herein as microtiter plates) typically have wells arranged in a regular 8×12 matrix configuration, or a multiple thereof (e.g., a 16×24 matrix configuration). In a 96 well plate, the center to center spacing between wells is roughly 9 mm. The microfluidic array may have individual channels that are between 5 microns and 100 microns across. Therefore, in some embodiments, solutions contained within the 96 wells of a multiple plate are to be delivered from a macrofluidic sample area of the multi-well plate of about 100 square centimeters to an analytical portion of a microarray and a reaction surface having as few as 10 square centimeters or fewer. To accomplish this, a transfer plate may be provided with a series of channels or fluid connections, as also referred to herein, each communicating between a channel on the microfluidic array and a well of the multi-well plate.

One desirable characteristic of the present invention is that using an assembly shaped like a standard multi-well plate allows the assembly to be used with existing laboratory equipment. The assembly can be fed into existing sample/reagent loading equipment, multi-well storage equipment. As such, no significant capital expenditures may be required to implement aspects of the invention. However, other configurations may be used as the invention is not limited in this respect.

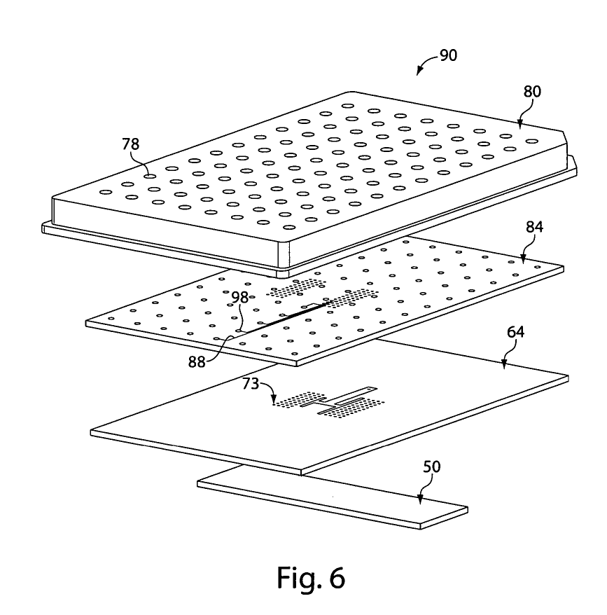

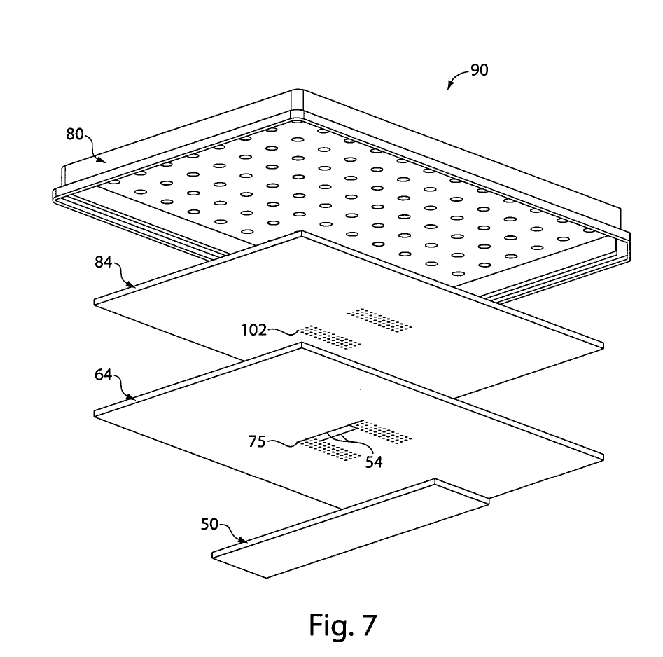

A top exploded view of an embodiment of a multi-well assembly is shown in FIG. 6 and a bottom exploded view of the same embodiment is shown in FIG. 7. In this embodiment, a transfer plate 84 provides fluid connections 88 that run from a bottom surface of the wells in the multi well plate 80 to individual channels of the microfluidic array 64. In some embodiments, these microfluidic connections may include a port or passageway that receives fluid again after it has passed through the microfluidic array, as aspects of the fluid connections are not limited to residing solely in the transfer plate.

In one illustrative embodiment, the fluid connections include a dead end type connection. In such embodiments, a channel begins at a well 78 of the multi well plate, passes through the transfer plate, traverses a channel of the microfluidic array where it terminates in a dead-end. In such embodiments, sample or reagent residing within the well may be driven through the fluidic channel, including the channel of the microfluidic array and any connected reaction surface, by pressure applied above the sample in the multi well plate. In some of such embodiments, any air trapped air in the channel may escape through the air-permeable walls of the microfluidic device or of other components in the assembly 90.

In one illustrative embodiment, the fluid connection may be a flow-through type fluid connection beginning at a well of the multi well plate and ending at a individual outlets or a common outlet for all of the channels. In some of such embodiments, sample or reagents in the wells of the multi well plate may be driven by pressure applied at the well of the multi well plate, such as light air pressure. In other embodiments, a vacuum at downstream ends of the channels, such as a common outlet, may be used to draw the sample or reagent from the well and through the fluid connection.

In one illustrative embodiment having a flow-through type connection, there are a limited number of outlets two (e.g., about two or three or four). This may greatly reduce the number of outlet ports that are needed in the microfluidic device and thus save valuable real estate in designing a system. Having a limited number of outlets also may prevent reactants from dwelling within the microfluidic channels. Systems that have a larger number of outlets may have problems with back flowing, because of surface tension effects at the channel inlets. Back flowing in some systems may allow contamination of the individual channels with reactants from other channels.

FIG. 31 shows a cross-section of an assembly with a sample plate connected to a transfer plate which in turn is connected to a microfluidic array sitting on top of a reaction substrate. In this embodiment, reactant provided within a well or guide of the sample plate is passed into a fluid connection 88 in the transfer plate. The reactant then follows the fluid connection until it reaches a transfer port, where it is passed into a channel of a microfluidic array. The microfluidic array exposes the reactant to a reaction substrate, as discussed herein, and then the reactant is evacuated out of an evacuation port. In this particular embodiment, the exhaust port extends back through each of the transfer plate and the sample plate, although other configurations are possible. As previously discussed, the various components of this assembly may be sealed against one another either with separate sealing elements, or by seal that is provided by the compliant nature of the components themselves.

Another illustrative embodiment of a fluidic connection includes a flow-through type fluidic connection. Here, the connection begins at a well of the multi well plate and ends at another well of the multi well plate after it has passed through a channel of the microfluidic array and any transfer plate that facilitates such a connection. Such an arrangement may allow alternating exposure of sample and/or reagent to the channels of the microfluidic array as they travel from one well to another. Such embodiments may also allow other different samples or reagents to be sequentially introduced into the channels for more complex assays. For example, in one of such embodiments, reagents for a first hybridization reaction may be followed by an introduction of a wash solution to remove unhybridized or unreacted reagents.

It is to be understood that any of the above described fluid connections, or others, may be incorporated into assemblies 90 like those illustrated in FIGS. 6 and 7 that may be used to deliver sample and/or reagent to a microfluidic array 64 and a reaction surface 50. FIGS. 6 and 7 show a 96 multi-well plate 80, a transfer plate 84, a microfluidic array 64, and a reaction surface 50 that are used, in combination, to deliver samples or reagents from the wells to the reaction surface through an array of microchannels, as previously described. The transfer plate layer may be used to provide a single layer of an assembly that is capable of routing all wells to a microarray configuration and a reaction substrate, or a subset of the wells, as the invention is not limited in this respect. The transfer plate layer may also prevent wells positioned immediately above the glass slide from inadvertently interacting directly with the slide.

The multi-well plate of the assembly may receive samples or reagents from standard laboratory equipment, in many cases through automated procedures. In one embodiment, the multi-well plate is a docking device described herein for simplifying fluid delivery to the transfer plate. The transfer plate may then deliver the samples or reagents to an array of microchannels formed in a microfluidic array such as those described herein. In many embodiments, the microchannels either deposit samples onto a reaction surface or pass reagents across previously deposited samples to perform an assay. In other embodiments, the microchannels pass samples across previously deposited reagents on the substrate surface. In general, different patterns of channels can be used to route fluids through the assembly. However, many patterns share the following common steps. Fluid is moved from a well in a multi well plate and routed towards the transfer plate, such as through an aperture or orifice in the bottom of wells in the multi-well plate. Fluid passes through the transfer plate toward the microfluidic array, the fluid then passes through the microfluidic array and contacts the reaction substrate.

In some illustrative embodiments, the fluid is moved through these channels as a result of a pressure differential applied to the fluid. This pressure can be delivered either in the form of a vacuum administered to the underside of the assembly. Alternatively, positive pressure can be applied to the top of the assembly. Still, in some embodiments the pressure differential may be created by a combination of applied vacuum and positive pressure.

In one embodiment, a vacuum may serve a second purpose in addition to pulling fluid through the microchannels. The vacuum may provide a suction force necessary to hold the assembly together (i.e. hold the multi well plate to the transfer plate, the transfer plate to the microfluidic array, and the microfluidic array to the reaction surface) and prevent leaking at the microfluidic array/reaction surface interface.

Vacuum systems are common on most high throughput screening robotic handling stations such as those manufactured by Beckman Coulter. The vacuum is made available for filtration operations. In standard practice, a gasket sized to interact with a multi well plate is stationed within reach of the robotic handling equipment. The plate is placed on the gasket and a tight seal is formed. A pump then pulls a vacuum on the space below the plate, establishing a pressure gradient from the atmosphere above, through the filtration plate, and into the evacuated chamber. The microfluidic process described here operates in a compatible way. Instead of simply filtering, the fluid is routed through intentionally designed microfluidic channels.

However, in some embodiments, a positive pressure of approximately 2-3 psi may be applied for approximately 5 minutes to drive reactants into the microfluidic channels. For example, in some embodiments nucleic acid probes are forced into dead-ended microfluidic channels to contact surface bound target samples for hybridization reactions. In these embodiments, a fixture or clamp is used to keep the components of the multi-well assembly together (the multi-well plate, the transfer plate, the microfluidic array, and the reaction substrate).

The Sample Plates

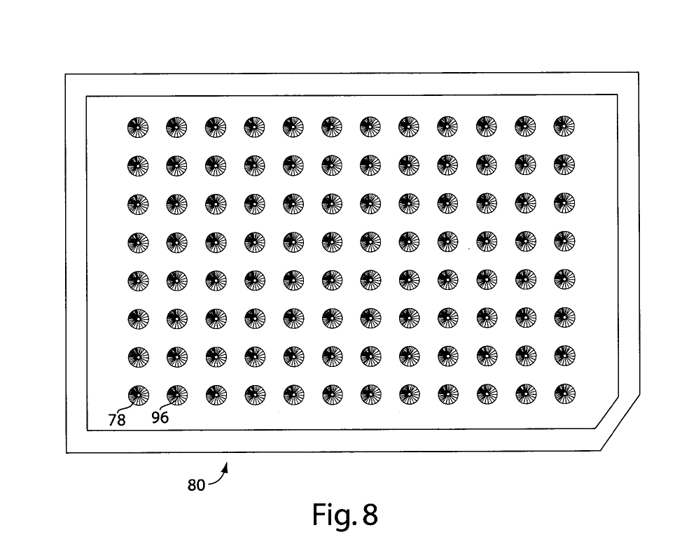

According to aspects of the invention, a reactant solution may be delivered to an array or transfer plate from a sample plate. A sample plate may be a multi-well plate such as one described herein. However, a sample plate may not be required. In other aspects of the invention, one or more reactant solutions may be loaded directly into a receiving port or inlet port on a transfer plate or an array using a dispenser such as a pipettor. In some embodiments, a docking interface of the invention may be used to deliver one or more solutions to a transfer plate or an array. The multi-well plate illustrated in FIGS. 6 and 7 has a 80 mm×120 mm footprint with 96 wells arranged in an 8×12 configuration, the wells having a 9 mm center-to-center distance. However, other standard multi-well plate configurations, such as a 384 well, a 1536 well, or any other plate configuration, standard or custom, may be used as the invention is not limited in this respect. According to aspects of the invention, a multi-well plate may have one or more openings (orifices) towards the bottom of one or more wells. In one embodiment, the multi-well plate also may have one or more exhaust through-holes such as the hole illustrated in FIG. 31. Samples or reagents may be deposited into each well of the multi-well plate and subsequently delivered through an orifice 96 in the bottom of each well 78, as shown in FIG. 8, to the transfer plate. In some embodiments, a multi-well docking device may be used as described herein. In one embodiment, the orifice in each well may be about 0.5 mm in diameter and is centered in the bottom of the well. However, other diameters, cross-sectional shapes, locations and sizes may be used as the invention is not limited in this respect.

The sample or reagent may be drawn into the transfer plate simply by gravity. Alternatively, other forces may assist flow into the transfer plate. In some embodiments, a vacuum assists or causes the sample to be pulled into the transfer plate. Vacuum sources may be used to pull all samples or reagents from the multi-well plate at once, or valve systems may be devised to pull samples from wells individually, when desired by a user. In other embodiments, a pressure may be applied across the top surface of the multi-well plate to force the samples or reagents to the transfer plate. As with embodiments using a vacuum to draw samples into the transfer plate, this can be accomplished for the entire multi-well plate assembly in the aggregate, or it may be applied to each well individually as desired. In still other embodiments, the samples or reagents may be moved from the multi-well plate and through the assembly by other means, such as with the assistance of a centrifugal force, or through electrical forces acting on the samples or reagents.

In some embodiments, the orifice is sized such that the surface tension of the reactant will prevent it from passing to the transfer plate from the well until a force is applied to the reactant. In such embodiments, pressure applied from above the wells or a vacuum applied through the transfer plate, or other forces as described above may cause movement of the sample or reagent instead of simply assisting its movement into the transfer plate. In other embodiments, a pin may be used to break the surface tension of the sample near the orifice of the well and thereby allow it to pass through the orifice into the transfer plate.

The assembly may be provided with a seal (not shown) to seal reactants in the wells before use or in between uses. Such a seal can be an adhesive seal or other type of seal. In other embodiments, a seal (not shown) may be placed over the top surface of the multi-well plate, or over the top surfaces of individual wells or sections of wells to help retain the samples or reagents within each well until the seal is removed. These seals can be useful to prevent fluid leakage or evaporation from a multi-well plate. The seal can include reusable components such as a plastic or rubber seals that mate with the top surface of the multi-well plate, or disposable components such as foil that adhere to the top surface of the multi-well plate or other devices, as the invention is not limited in this respect.

The ability to seal samples or reagents within a multi-well plate allows the multi-well plate to be provided prepackaged with samples or reagents. It may be desirable to perform an assay against a known sample, for experimental control or other reasons. Also, it may be desirable to provide a multi-well plate with a predetermined combination of reagents, such as probes, for a common type of assay. To this end, having the ability to seal contents within the multi-wells allows a multi-well plate to be provided ready to mate with a transfer plate and/or reaction substrate to perform an assay.

The multi-well plate is preferably injection molded out of plastic material and can be molded with the orifices in each well, or they may subsequently be added by drilling, punching or other known manufacturing processes.

The Transfer Plate

The transfer plate of the multi-well assembly delivers reactants from a multi-well plate designed to interface with conventional laboratory equipment to a reaction surface that maximizes assay reaction density. Multi-well plates, whether they are standard configurations, such as a 96 well, 384 well, 1536 well configuration or a custom configuration, are generally adapted to interface with conventional laboratory equipment. The microfluidic array, as previously described, allows many assays to be produced within a very small area of a reaction surface. The transfer plate provides an interface between the macro-scale multi-well plate and the micro-scale microfluidic array, without adversely impacting the efficiency associated with conventional laboratory equipment.

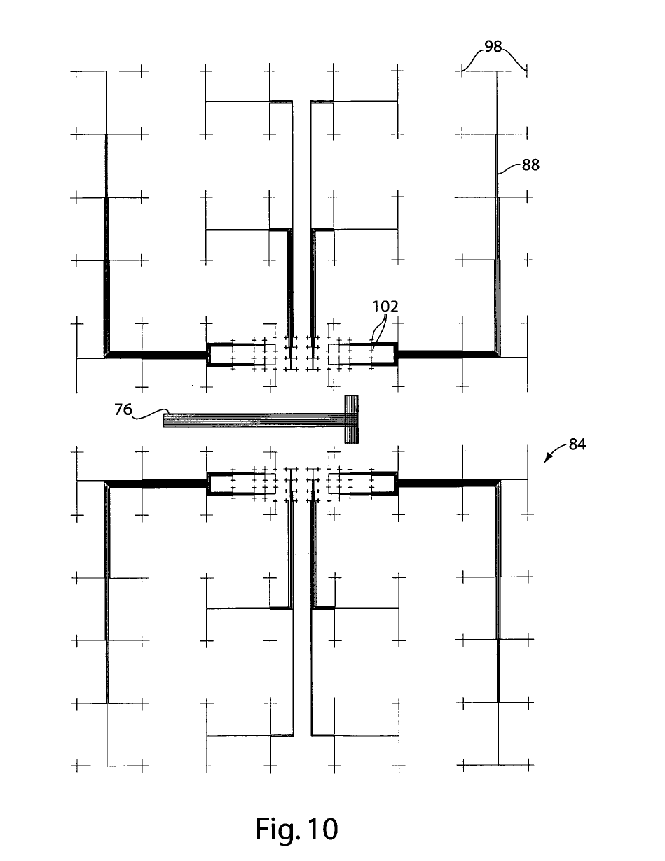

To deliver reactants from a macro-scale environment to a micro-scale environment, the transfer plate, as depicted in FIGS. 10, 13, and 31 first accepts each reactant through the orifice in each well and into the first receiving end 98 of a transfer channel 88. In the illustrations, the transfer channels and the channel receiving ends, marked with a cruciform in the drawings, have the same cross-sectional dimensions. However, in other embodiments, a channel receiving end may include a larger cross-sectional area to help insure fluid communication with the orifice in each of the multi-wells. In particular, having a receiving end of a larger area may make the design more tolerant to manufacturing variability in the location of the orifice of each well, or to the location of the receiving end of each channel. The receiving end of each channel may include a through hole or a blind hole. However, blind holes are preferred, at least for channel ends that will be placed directly above the microfluidic array. Using through holes in such areas may result in leakage near the microchannels of the microfluidic array.

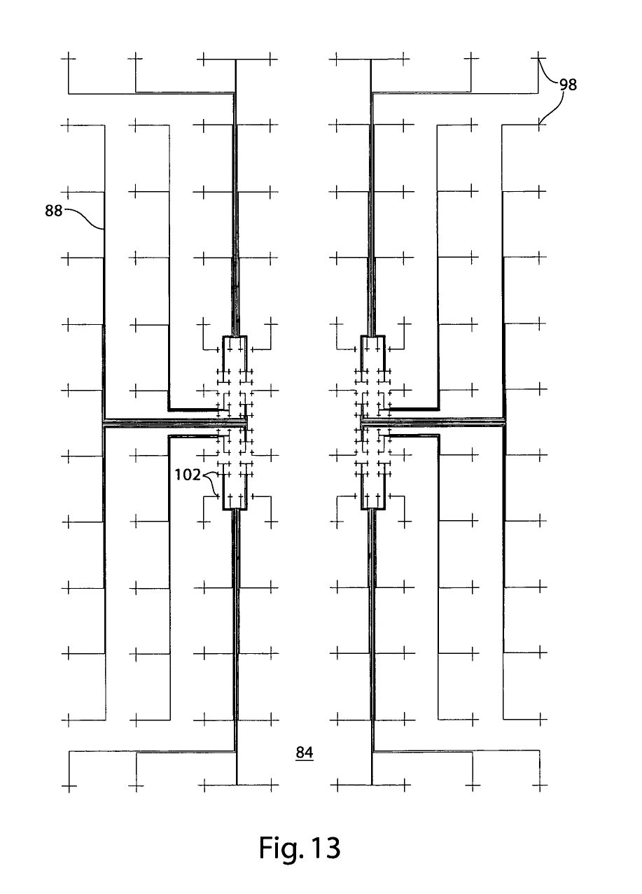

The channels of the transfer plate may follow any path from each of the receiving ends to each of their respective transfer ports 102 at the opposite end of each transfer channel, which are used to transfer the reactant to the microfluidic array. FIGS. 10 and 13 show a top view of a transfer plate. It should be appreciated that the receiving end 98 is open to the upper surface of the transfer plate, whereas the transfer port 102 is open to the lower surface of the transfer plate. The transfer channel may connect the receiving end to the transfer port following any suitable path such as those described herein. In some embodiments the channels may be standard lengths, to the extent possible. Matching the length of each of the channels may generally provide every reactant with a similar distance to travel. This may be preferred in some assays in order to provide consistent test conditions for each reaction. In other embodiments, channels that have a shorter length may be restricted in some other manner to help equalize the time it takes a given reactant to flow through a channel. Reducing the cross sectional area of a portion of a channel, or an entire length of a channel, is one way to create such a restriction. In one embodiment, the transfer channel(s) may be enclosed within the transfer plate. In another embodiment, the transfer channel(s) may include an open channel on the upper surface of the transfer plate that is closed when the sample plate (e.g., multi-well plate) is placed on top of the transfer plate. In yet another embodiment, the transfer channel(s) may be located on the bottom of the sample plate. Accordingly, in one embodiment, the sample plate and or transfer plate are made of a suitable material and design to form a seal when the sample plate is placed on the transfer plate. In one embodiment, the sample plate has a flat or substantially flat lower surface. In one embodiment, the transfer channel(s) may be 100 microns deep by 100 microns wide. However, other sizes may be used.

It is also preferable to direct all of the channels such that their transfer ports exist in two separate groups having an unoccupied space located between the two separate groups. Such arrangements present an efficient arrangement of transfer ports and corresponding inlet ports on the microfluidic array. The unoccupied space between the areas also may be an efficient spot to locate exhaust channels or ports 76, as shown in FIG. 10. However, exhaust channels or ports also can be located at other positions on the transfer plate, or even in other portions of the assembly, such as on the microfluidic array.

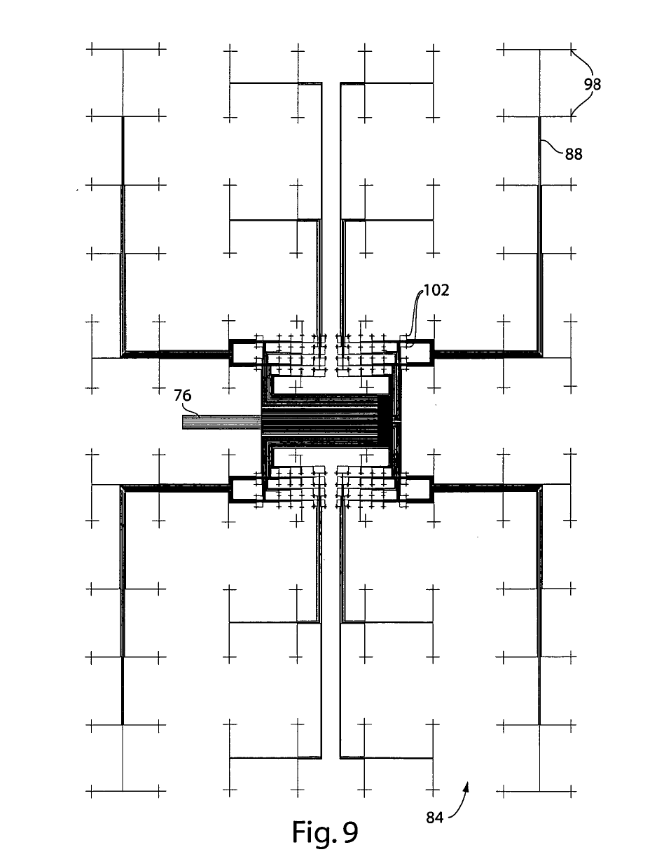

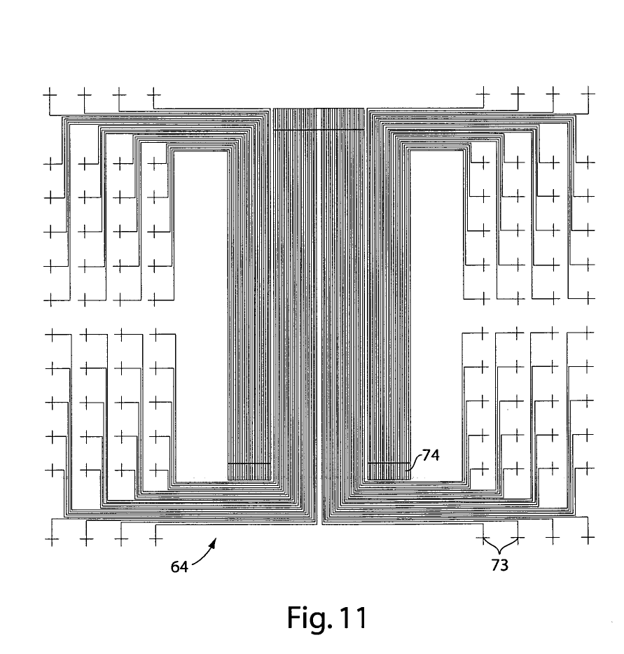

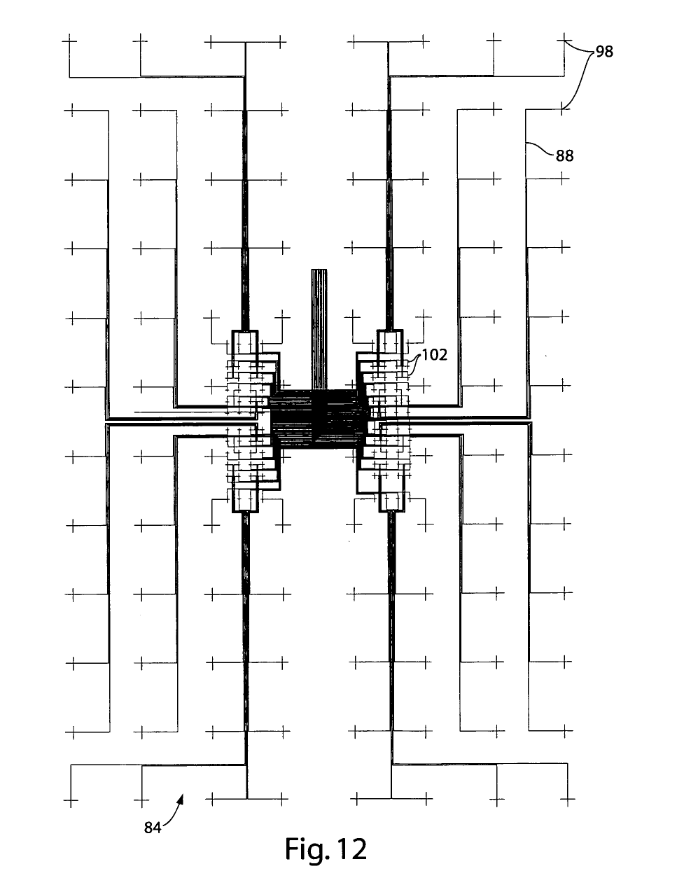

In one embodiment, exhaust channels are included in the transfer plate of FIG. 10 for receiving reactants (e.g. samples or reagents) once they have passed through the microfluidic array shown in FIG. 11. It should be appreciated that the microfluidic array is shown through a top view and that the inlet ports are open to the upper surface of the array and connect through the array to the channels that are on the lower surface of the array. Similarly, the exhaust ports connect the channels on the lower surface of the array to the upper surface of the array. The microfluidic array shown in FIG. 11 also may be referred to as a print head, because it may be used to print an array of reactants (e.g. samples or reagents) on a reaction surface. In operation, reactant solutions are flowed through the microfluidic array, across the reaction surface, and into the exhaust channels as discussed herein. FIG. 9 shows a top view of the transfer plate of FIG. 10 positioned over the array of FIG. 11 with the transfer ports of the transfer plate aligned with the inlet ports of the array and the exhaust channels/ports of the array aligned with the exhaust channels/ports of the transfer plate. The exhaust channels may comprise a single, common channel having one return port in communication with the entire plurality of microchannels in the microfluidic array, a plurality of individual microchannels each in communication with an independent exhaust channel at their own respective return port, or any combination of microchannels in fluid communication with any group of exhaust channels.

The exhaust channels shown in FIG. 10 terminate in a common, main exhaust port that extends from the transfer plate, through the microfluidic array and out of the assembly. This exhaust port may be placed in fluid communication with a vacuum pump to pull reactant through the assembly, alone or in combination with other means for directing reactant through the system. In particular, assemblies having such a main exhaust port on their bottom surface can be placed over a vacuum block to provide a suction force for moving the reactant through the channels of the assembly. In other embodiments, particularly where the reactants are driven through the assembly by pressure applied over the top of the multi-well plate, the main exhaust port may serve only as passive exhaust for used samples and reagents. Although the exhaust port is shown extending out of the bottom surface of the assembly, other embodiments may have an exhaust port located in other positions as the invention is not limited in this respect. In one embodiment, one or more common exhaust ports may connect to the exhaust channel(s) and extend through to the upper surface of the array, through the transfer plate and through the sample plate. Such exhaust port(s) also may serve as a passive or active exhaust (e.g., a vacuum may be applied to the exhaust port(s)).

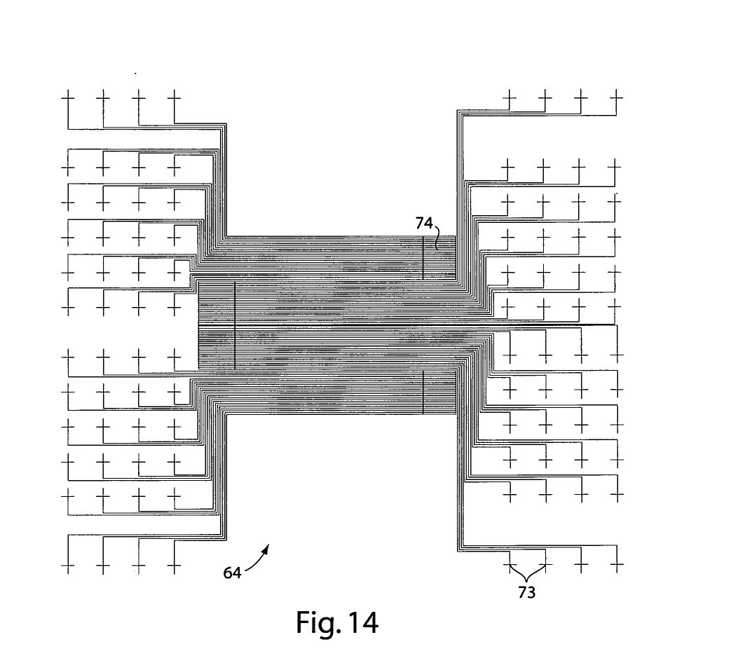

In contrast, the transfer plate shown in FIG. 13 does not includes an exhaust channel. The transfer plate of FIG. 13 may be used in conjunction with a microfluidic array containing dead-end microfluidic channels 77, such as the microfluidic array of FIG. 14. Such a microfluidic array may also be referred to as a hybridization head, because it may be useful to deliver a plurality of reactant solutions to a reaction surface that already contains immobilized reagents. In most of such embodiments, a positive pressure force is applied to the top of the multi-well assembly. This positive force drives air and the sample or reagent through the wells, into and through the transfer plate, into microfluidic array where the channels come to a dead end. The fluid, typically air, that is trapped and compressed as the reactant is pushed through the assembly escapes by diffusion into the porous walls of the channel. In other embodiments using dead end flow, the exhaust channels may be lengthened or shortened to alter the flow characteristics of the assembly. Embodiments having longer channels downstream of the microfluidic array will generally allow more driving fluid, such as air, to drive sample or reagent through the microfluidic array.

The transfer channels and exhaust channels of the transfer plates depicted in the drawings may be about 50 microns×10 microns and have about a 500 square micron cross section, although in other embodiments the channels may have other cross sectional shapes, dimensions or minimum spacing between channels as the invention is not limited in this respect.

The transfer plate may be provided to receive reactants from a multi-well plate and to deliver them to a microfluidic array. The transfer plate may also provide a convenient location for an exhaust port. As such, the transfer plate may comprise a different design that accomplishes one or more of these effects.

The transfer plate, in some embodiments, is manufactured of polyurethane or PDMS and may be made through a soft lithography process. Preferred materials are generally inert and thus do not interfere with the samples or reagents or their passage through channels in the transfer plate. These materials may also have natural porosity levels that are suitable for embodiments that use the dead end flow technique described above. Additionally, these materials are typically soft enough to form an sufficient seal between the microfluidic array and the multi-well plate obviating the need for an additional sealing material to be included in the assembly, although some embodiments may include sealing material to improve the seal between any of the components of the assembly.

Although soft lithography may be used, other manufacturing processes also may be used and may present certain advantages for certain uses. Photolithography techniques may be employed to manufacture transfer plates. Additionally, for some embodiments, particularly those employing transfer channels of larger dimensions, other manufacturing processes may be employed, such as machining. Other techniques may include standard plastic molding techniques such as those used to mold diffraction gratings Plastic molding techniques may include poured molding and/or injection molding techniques. Molds may be etched or formed using any technique as the invention is not limited in this manner. It should be appreciated that any of the materials and manufacturing techniques described herein may be used for any of the aspects of the invention, including, but not limited to, a reaction substrate, a microfluidic array (e.g., a print head or a hybridization head), a transfer plate, a reactant solution reservoir, a docking device, a sample plate or other component of the invention.

The transfer plate depicted in the figures may be a separate component of the multi-well assembly that fits into a cavity in the bottom of the multi-well plate. Although not shown, the transfer plate may have registration features, similar to the truncated corner of the multi-well plate, that help a user assemble the device properly. In addition to registration features like the truncated corner that helps orient the transfer plate in the correct rotational position, the interface between the transfer plate and the multi-well plate also may include registration features that insure the top side of the transfer plate (and not the bottom side) is assembled against the bottom side of the multi-well plate.

A sample or reagent plate such as a multi well plate may be made of hard plastic such as polystyrene, polycarbonate, or polypropylene. In some embodiments, the lower surface of the multi-well plate can be embossed or otherwise modified to have an array of channels adapted to connect to the microfluidic array. This may eliminate the need for a separate transfer plate in some embodiments.

The Microfluidic Arrays

Microfluidic arrays of the assembly include the print heads and hybridization heads described above. The microfluidic array shown in FIG. 11 may be a print head adapted to return reactants or driving fluids back to the transfer plate for exhausting. However, in some embodiments, the print head may contain a direct exhaust, as the invention is not limited in this respect. Still, in other embodiments some features of the microfluidic array may be included in the transfer plate. However, to simplify manufacturing in some embodiments, it may be preferred to make the transfer plate and microfluidic array as separate components.

The microfluidic array shown in FIG. 11 may be used as a print head for depositing sample onto a reaction surface, while the microfluidic array shown in FIG. 14 may be used as a reagent head or hybridization head for passing reagents transversely over previously deposited samples. Thus, in operation, the microchannels shows in FIGS. 11 and 14 run in directions that are perpendicular to one another to allow the previously discussed assay matrix to be formed on a reaction surface without changing the relative position of any of the other components of the assembly. However, either of these arrays may be used for either printing or reaction procedures.

Like the transfer plate, the microfluidic array may be sized to fit within the cavity on the underside of the multi-well plate, along with the transfer plate. In one embodiment, the bottom surface of the microfluidic array may be flush with the bottom surface of the multi-well plate when assembled. In such embodiments, an exhaust port on the bottom surface of the microfluidic array may be configured to readily seal with a vacuum block placed beneath the assembly.

Although not shown, some embodiments may include a recess in the bottom surface of the microfluidic array for accepting a standard, glass or silicon test slide as a reaction surface/substrate. In these embodiments, the reaction surface and other components of the assembly may be placed flush against a vacuum plate to create a seal between any exhaust port on the microfluidic array and to support the slide and the other components of the assembly with only the flat surface of the vacuum plate. In other embodiments, two different recesses may be formed in the bottom surface of the microfluidic array to accept a slide in a first position for depositing samples on the slide, and in a second position for running reagent across the samples in a transverse direction. Like the interfaces between the transfer plate and the multi-well plate, the interface between the reaction surface and the microfluidic array may have registration features that only allow the reaction surface and the microfluidic array to be interfaced in proper orientations. Such registration features may include a truncated corner or corners, one or more pins, ridges, or grooves, or any other features that help guide the interface between the microfluidic array and the reaction surface, as the invention is not limited in this respect.

In one embodiment, the microfluidic array may be bonded to the transfer plate to ensure proper alignment between the transfer ports of each component. In another embodiment, the multi-well plate may be bonded to the transfer plate and the transfer plate may be bonded to the microfluidic array to form a single transfer system. These components can be permanently joined, bonded, sealed, or affixed using known manufacturing methods. Alternatively, any combination of these components may be temporarily bonded so that they may be provided to an operator in a preassembled form at with the appropriate holes suitably registered. After use, an operator may remove the reaction substrate and process it to detect any reaction signals of interest.

However, in other embodiments, the transfer plate and the microfluidic array may be separate components. As with other components of the assembly, the transfer plate and the microfluidic array may include features that help align them with respect to one another. Such features can include one or more alignment pins, ridges, or grooves, or other features as the invention is not limited in this respect.

The microfluidic array may be made of polyurethane or PDMS material and may be made through a soft lithography process associated with such materials. These materials are generally chemically and energetically inert with respect to the samples and reagents that pass through the microfluidic array, which may be preferred. Additionally, when the microfluidic array is used in combination with a glass or silicon reaction surface, like most standard slides, the energetic attraction between samples or reagents and the reaction surface may not be disturbed.

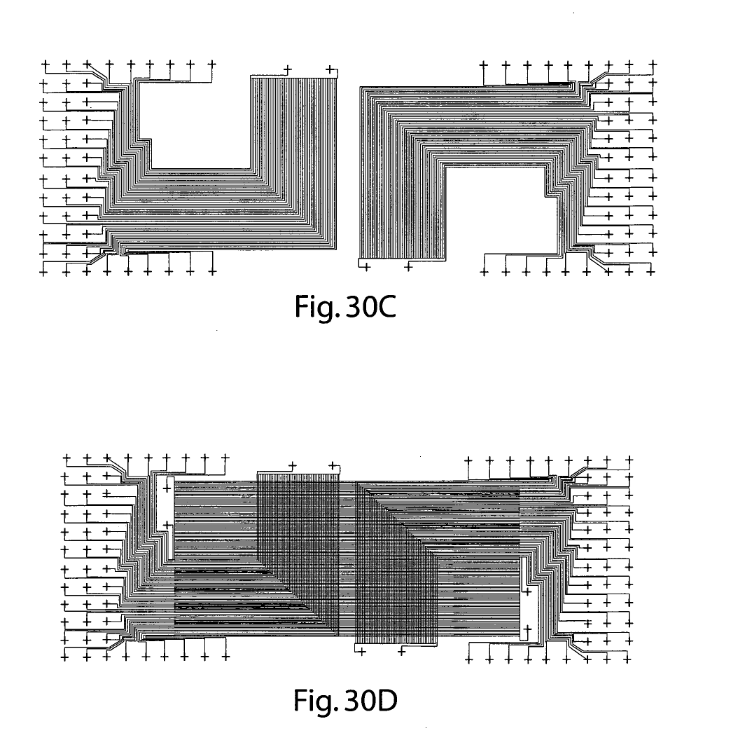

In some embodiments, the microfluidic channels in a microfluidic device may be organized into two subsets that flow in opposite directions in parallel paths. This is illustrated in FIG. 14 where half of the channels flow in one direction and the other half flow in the other direction. In this configuration, half of the channels are arranged in a single central bundle that flows in one direction. This central bundle is intercalated between two outer bundles of channels that flow in the opposite direction from the channels in the central bundle. This configuration and similar configurations involving subsets of channels flowing in opposite directions (see for example FIG. 11) are used to fit a large number of channels connected to a two-dimensional array of sample wells onto a relatively small surface. However, any configuration of microfluidic channels and/or transfer plate may be used as the invention is not limited in this respect. Other illustrative configurations are shown in FIGS. 30a-30d.

The invention therefore provides a relatively inexpensive platform that can be adapted to fit existing automated equipment and that can be used to perform large numbers of assays. The automation platform needed to use the microfluidic systems of the invention may be similar to the complexity of a microarray printer which consists of plate hotels, a robotic manipulator, and a flat bed that holds the glass slides. Detection of hybridization events may be possible using a standard microarray reader, similar to that used for detecting the labeled probes in the Examples. Standard glass microscope slides can hold 1 of 1536 lines by 3 of 1536 lines, where the channels are 10-microns in diameter. This may provide for 7 million spots per slide, that can be autoloaded from just 4 of 1536 well multi well plates. Furthermore, both samples and probes can be presented to the array of microfluidic channels using standard multi-pipettors, drawing samples from standard multi well plates. However, customized equipment also may be developed and used in certain aspects of the invention.

Interface/Docking Device

Other aspects of the invention are directed to improving the interface between an assembly (e.g., a microfluidic array alone, or a microfluidic array associated with a transfer plate) of the invention and laboratory equipment, such as pipettes or multi-pipettors, pipette tips, deposition needles, or multi-well plates. As is to be appreciated, the small volume of reactants that are deposited into wells of a multi-well plate may exhibit relatively strong surface tension characteristics. Such characteristics may allow the reactant to adhere to a side wall of a sample well, or another portion of the sample well other than the aperture in fluid communication with the microarray (optionally via a transfer plate). In this regard, the reactant may not pass toward the microarray, but rather remain within the well. As is to be appreciated, this is not a desirable trait in many embodiments.

To address such issues, attempts have been made to precisely position the multi-well plate with respect to pipettes or other dispensing instruments prior to reactant being dispensed into the wells. However, such techniques are not always effective at guiding reactant to a well, or to a desired position within the well, particularly where hand dispensing techniques are used. The reactants, when using these techniques, tend to either stay on the tip of the dispensor or stay on the walls of the sample well, never reaching the target within the well, such as the previously described orifice. Additionally, techniques used in the prior art may have difficulty dispensing the reactant to the well without motion of the dispenser 104 or direct contact with a sidewall of a well. These aspects of dispensing may allow the dispensed reactant to miss a target within the well. Accordingly, such techniques may not be suitable for some applications of the invention where a small amount of solution is to be delivered to a microfluidic array either directly or using a transfer plate according to aspects of the invention.

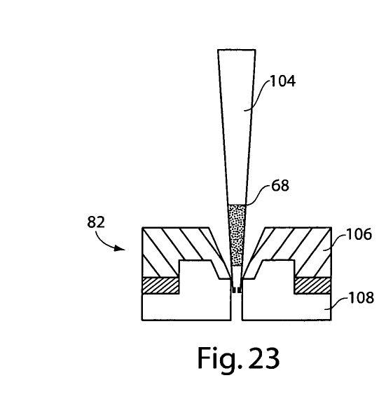

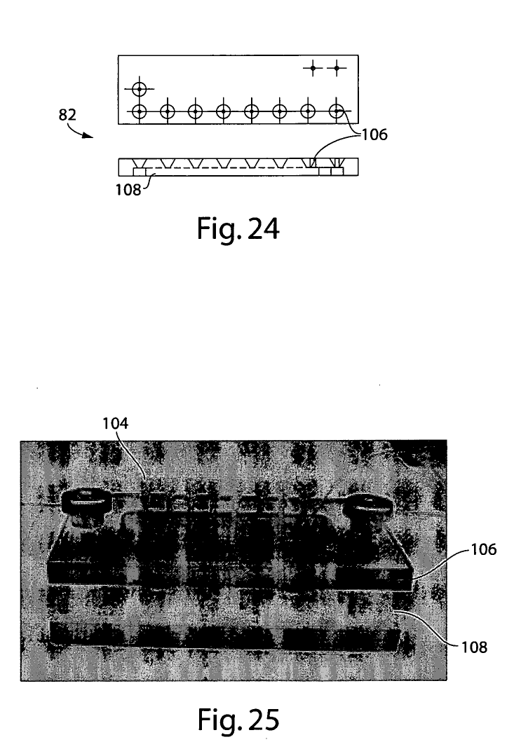

As illustrated in FIGS. 23 and 24, one embodiment of the invention includes a docking interface 82 that may facilitate delivery of reactant solutions 88 from wells 78 of a multi-well plate, either directly into microfluidic channels of an array (optionally into channels of a transfer plate that is in fluid communication with the array). As illustrated, the interface may include a guide plate containing one or more guides 106 adapted to align a tip of a dispenser 104, such as a pipette, with a target in a well, such as an orifice at the bottom of the well. In some embodiments, the guide may be the walls of the well. In other embodiments, the walls of each well in a multi-well plate may be adapted for guiding a dispenser tip to an opening at the bottom of the well. In other embodiments, an adaptor may be provided to more precisely guide each dispenser tip. The interface may also include a retainer 108 to hold the dispenser tip in alignment with the target when the reactant is dispensed toward the target. According to aspects of the invention, the target may be a receiving or inlet channel or port in either a transfer plate or a channel array.

The guide plate shown in the illustrated embodiment includes funnel shaped portions that share a common center-to-center spacing with wells of a mating multi-pipettor. Each of the funnel shaped guides may be used to guide a dispenser accordingly. As is to be appreciated, in some embodiments the guide plate may include the same number of funnel shaped portions as the number of tips on a one-dimensional or two-dimensional pipettor. However, the invention is not limited in this respect, as the guide plate may have any number of individual guiding elements, such as fewer than a multi-pipettor or multi-well plate. It should be appreciated that the guide plate may include any number of guides (from 1 to several thousand). The guides may be aligned in a single dimensional array adapted to receive and guide the tips of a one-dimensional multipipettor (e.g., 4, 6, 8, 12, 16, 32, or other number of tips). The guides may be aligned in a two-dimensional array adapted to receive and guide the tips of a two-dimensional multipipettor (e.g., 64, 96, 384, etc.).

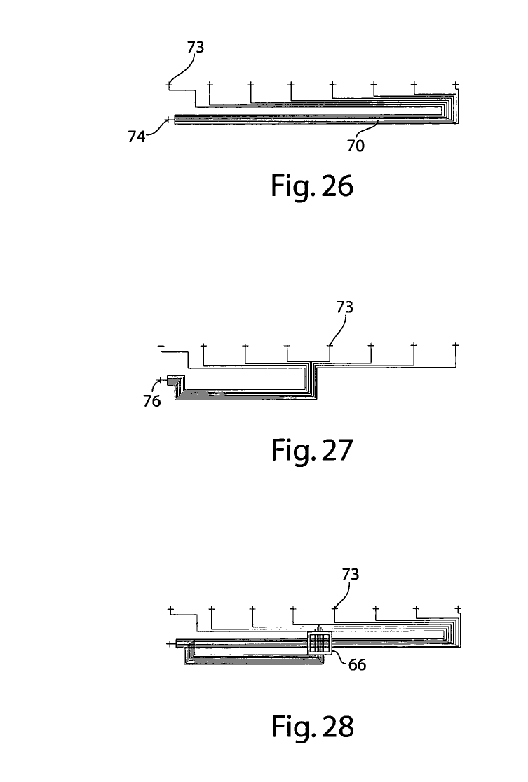

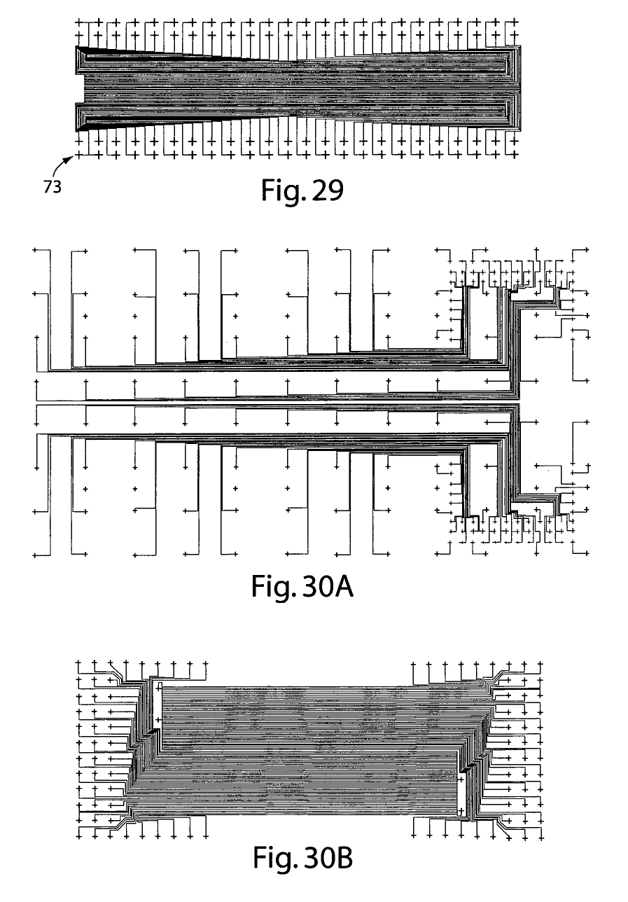

In one illustrative embodiment, the guide plate mates with an upper surface of a transfer plate having channels that direct reactants toward a channel array that may be mated with the lower surface of the transfer plate. In another embodiment, the guide plate mates directly with the upper surface of a channel array (the surface that is opposite the channel presenting surface of the array). By way of example, FIGS. 26 and 27 each show a plate that may be used with an interface device to direct reactant directly to a microfluidic channel used to deposit reactant onto a reaction surface or to flow reactant over previously deposited reactant to perform an assay. FIG. 28 shows an overlay of the plates of FIGS. 26 and 27 showing how they overlap in a particular zone where interaction surfaces or contact points are created. FIG. 29 shows a top view of an embodiment of an array with 96 inlet ports 73 on an upper surface connected to an array of open channels on a lower surface. In this embodiment, the arrangement and spacing of the inlet ports would not accommodate a delivery device such as a multi-pipettor. Accordingly, this array should be used with a transfer plate.

The guide plate may mate with a transfer plate or channel array in a variety of different manners. In one embodiment, the guide plate is adapted to be clamped against a channel array (such as the embodiment of FIG. 25, which is shown held within a clamping fixture) while in other embodiments the guide plate may be designed to sit on top of a channel array or transfer plate without the assistance of any mechanical clamps. In one embodiment, the guide is immobilized on the underlying plate or array by applying a vacuum (this may be the same vacuum that is used to draw reactant solution into the channels in some embodiments). In some embodiments, the guide plate may mate directly to the underlying device while in other embodiments, the guide plate and the multi-well plate may be separated by other components, such as a seal, as is described in greater detail below. Still some embodiments may incorporate alignment features between the guide plate and the attached device(s) to insure proper assembly. These alignment features may include alignment pins, notches, matching truncated corners, or other features as the invention is not limited in this regard.

In one illustrative embodiment, a seal may be disposed between the guide plate and the device to seal the tip(s) of installed pipettes against the ambient atmosphere. In other embodiments, the seal may provide a sealed passageway that helps direct reactant within the dispenser toward the target channel or inlet. Still, in some embodiments, the seal may allow the contents of the dispenser to be drawn toward the target by the application of a differential pressure at the tip of the dispenser. By sealing the passageway against ambient atmosphere, substantial leaks into the well and/or other portions of the fluidic connection may be prevented, thus allowing the sample to be efficiently drawn in toward the target (e.g., using a vacuum or by applying positive pressure). In embodiments where the guide plate and seal mate directly with a transfer plate or array of microchannels, the passageway may include an enlarged area on its mating side that helps ensure engagement with channel of the transfer plate or an inlet port of a channel in an array. Alternatively, or in addition, an enlarged area may be included around the inlet hole or channel on the surface of the transfer plate or microchannel array that is in contact with the orifice(s) in the guide plate (or that is in contact with the orifices in the seal).

In some embodiments, the retainer may act as the seal. Accordingly, in one embodiment, a guide plate may include a guide and a retainer that acts as a seal, with no separate retainer. In one illustrative embodiment, the seal comprises a sheet of compliant material that may be placed between a guide plate and a device. Here, the seal may include a plurality of through holes, each aligned with a target in a corresponding well of a guide plate such as a multi-well guide plate of the invention. The holes have diameters sized to accept dispensers commonly used in laboratory automation, and may be sized to provide an interference fit between with the dispensers to effect the seal there between. As is to be appreciated, the seal is not limited to be a sheet of material as described in with respect to this embodiment, as the seal may be configured in other manners as those of skill will appreciate. For example, the seal may have features that align it with either the guide plate or the underlying device. The holes within the seal may include other features, such as a counter bore on the well side of the seal to allow for better aspiration of reactant. Still, other embodiments may be adapted to mate with only a portion of a guide plate instead of the entire plate. In some embodiments, the seal may be adapted to reside in a seal groove of a guide plate, while in other embodiments, the guide plate and the seal may be a unitary element.

As previously mentioned, the interface may include features to retain the dispenser in alignment with a target in a corresponding well. In some embodiments, the compliance of the seal itself may hold a dispenser in alignment with the target. In some embodiments, a pipette tip may be lodged by the lab equipment within portions of the seal that are associated with a first subset of the wells, the lab equipment may then be used to lodge additional subsets of pipette tips within the seal until all seals are filled. Differential pressure, such as a vacuum, may then be applied to the device to simultaneously draw reactants from all of the tips into corresponding targets of the wells.

The various components of the interface may be made of any materials know to those of skill. In one embodiment, the seal is made of a compliant material, such as silicone, RTV, or PDMS. In one embodiment, the guide plate is made of a plastic, such as polypropylene, nylon, or ABS plastic, to name a few non-limiting examples. Additionally, components of the interface may be manufactured through any procedure known to those of skill, including but not limited to, injection molding, soft lithography, machining, and any other suitable forming process as aspects of the invention are not limited in this regard.

Improved Reaction Time

As mentioned herein, traditional microarray systems involve immobilizing reagents onto glass slides by depositing droplets of solution filled with DNA on the surface and letting the droplet dry to leave behind the DNA. In such scenarios, the amount of DNA deposited on the surface may be equal to the amount of DNA that was in the droplet.

In one illustrative embodiment of the invention, each hybridized probe may be exposed to a significant portion of the sample (e.g., up to 100% of the sample), which may result in a much faster hybridization time—in some cases nearly instantaneous. It is to be understood that the concepts may apply equally as well to other reactions, such as protein-protein interactions or other reactant interactions, as aspects of the present invention are not limited to hybridization reactions alone. The improved reaction times may by accomplished with a microfluidic channel, like those of the microfluidic array in combination with a reaction substrate having immobilized reactant thereon, as discussed herein. The channel may be placed over immobilized reagent, such that the width of immobilized reagent on the reaction substrate is from 20% to 100% of the width of the channel.

As sample is flowed though the channel and over the immobilized reagent, each of the targets contained within the flowing sample are sequentially brought close to the immobilized reagent, within at most the height of the channel, measured vertically from the reaction surface. This may allow a majority, if not all the DNA targets in the sample to diffuse a very short distance to hybridize to the reagent of the reaction substrate. In this regard, substantially increasing the number of hybridizations that occur. Similar results may be accomplished for reactions other than hybridization reactions. This, in turn, may increase the detectability of the signal resulting from the hybridization, which may improve the accuracy and quality of the assay being performed. For small channels with cross sectional distances in the 10 micron range, the amount of target that can diffuse to the wall may be approximately 20% or more of that which is contained in the flowing sample. This may be 200 times the fraction of the sample targets that may be hybridized or even reached by corresponding reagents in conventional, stationary diffusion techniques. By way of example, a sample volume of approximately 1 microliter may be passed through a microchannel and exposed to an immobilized reagent, such as a probe, in approximately 5 minutes, versus the typical 10 hour exposure of techniques used in stationary hybridization techniques. As a result, the increased hybridization signal may be 200 times stronger than a non-flowing sample exposed to the same spotted probe for approximately 10 hours.

In one illustrative embodiment, the channel may be arranged such that the sample flows over sequential spots of immobilized reagent on the surface of the reaction substrate, where each spot contains a different type of probe. Here, each spot may experience the same high target DNA diffusion rate from the flowing sample.

In one illustrative embodiment, the microfluidic channels used may be from 0.01 to 0.02 mm high by approximately 0.04 mm wide. The sample velocity may be about 0.4 centimeters per second. Total flow times for either printing reagent onto a reaction surface or hybridizing sample DNA with the immobilized reagent may be between 3 and 5 minutes for a sample volume of about 500 nanoliters delivered to the reaction substrate. In some embodiments, the ability to identify 10 picomole concentrations of a specific target in a sample where other targets were present in the sample is possible. Here selective detection of 100 picomolar and 10 picomolar concentrations may be accomplished.

As is to be appreciated, when the concentration of a known analyte in a sample volume is measured, a general goal may be to obtain high measurement sensitivity using reasonable sized sample volumes. That is, it may be desirable to detect and quantify the smallest concentrations of specific analytes in samples that are as small in volume as practicable. In many scenarios, only small volumes may be available, and it may be necessary to have instruments as efficient as possible to be capable of detecting these small concentrations. Alternatively, larger samples may be concentrated by removing water, thus increasing the concentrations of the analytes and better enabling the instrument to detect them. Also, the cost of processing a sample for measurement may be high, and reduced sample volumes may be less costly to process.

In one illustrative embodiment of a biologic hybridization device, a camera is focused on a spot where hybridization between sample and reagent may have occurred to measure the signal intensity over an analytical portion of a substrate. This signal intensity may directly related to the number of labeled sample targets per unit area that hybridize to a complimentary immobilized reagent (e.g., probes) that are a reaction surface. The Detection Efficiency of a hybridization device may be described as proportional to the number of sample labels per unit area that are hybridized to a reagent, divided by the total number of targets that are available for hybridization with the reagent in the sample volume. As is to be appreciated, it may be desirable to have a higher detection efficiency to improve the quality and timeliness of assays that are performed with the instrument. High detection sensitivity may be reached when all the available labeled targets in the sample volume are concentrated on the smallest possible probe surface area that can be measured by the instrument camera.

It is to be appreciated that, for a sample that flows through a hybridization device, the number of labels hybridized to a probe in the device may be equal to the change in label concentration of the sample as it flows through the device. The total number of labels that are available to hybridize is equal to the total concentration of the sample, such that the Detection Efficiency may be expressed as shown by Eq. 1 below:
DE=(Ci−Co)/(Ci Ah) Eq. 1.
Where:
   Ci and Co equal the inlet and exit concentrations of reactant (e.g., 1 labeled target), respectively, and
Ah equals the hybridization probe area

Detection Efficiency may be measured for a given device where the entering and exiting concentrations of label are measured. For similar devices having similar mass transfer characteristics, such as channels with probes printed on the bottom of the channel, it may be appreciated that the channel with the smallest printed hybridization surface area may have the a higher efficiency. For example, one channel with half the width of a second channel may have twice the Detection Efficiency. In this regard, the narrower device may have twice the sensitivity to detect smaller concentrations of target in similar sample volumes, or the narrower device can be used with half the sample sizes to achieve the same concentration detection sensitivity.

In one illustrative embodiment of a substantially straight microfluidic channel, the Detection Efficiency may be closely approximated by Eq. 2 below.
DE=1.8/wx((x/d)/(ReSc))^{2/2} Eq. 2
Where:
   d and w are the height and width of the channel, respectively
   x and w are the length and width of the immobilized reagent, respectively
   Re is the Reynolds number based on channel height, and
   Sc is the Schmidt number.

According to aspects of the invention, the removal efficiency, RE, is equal to the fraction of labels removed from the sample volume as it flows through the microfluidic device. These labels may be removed from the sample because they hybridize to an immobilized probe area. The Detection Efficiency, DE, is the RE divided by the area of the immobilized reactant.

The actual intensity measured by a microarray reader is proportional to the number of hybridized labeled targets per unit area of hybridization probe area, or the L/A, as described in equation 3:
L/A_{—}flow=DE(V_{—}sample)(Ci) Eq. 3

Where V_sample is the total volume of sample drawn through the microfluidic device, Ci is the inlet concentration of reactant.

This equation demonstrates that the strongest signal is detected when DE is the largest. It also shows that for a given microfluidic device resulting in a specific DE, and a given minimum L/A that can be detected by a microarray reader, the product of V_sample and the concentration of the unknown target in the sample is equal to a constant. Therefore either more sample volume can be used to detect a weaker concentration of sample target, or less sample volume can be used to detect a stronger concentration of a sample target. In one aspect of the invention, a DE (DE=RE/area of reaction) is greater than 125, preferably greater than 200; more preferably greater than 500, more preferable greater than 1,000, more preferably greater than 2,500, and more preferably greater than 5,000 microns⁻². A high DE may be achieved by using small deposition and reaction channels. For example, channel widths (for either deposition methods, reaction methods, or a combination thereof) may be less than 100, preferably less than 50, more preferably less than 10, and more preferably less than 5 microns. Similarly, channel depths may be less than 100, preferably less than 50, more preferably less than 10, and more preferably less than 5 microns.

It should be appreciated that combinations of one or more devices or structures described herein may be assembled from individual devices or structures and provided as an assembly. However, in other aspects, combinations of one or more devices or structures may be provided as a single component device or structure. Also, in some embodiments a device or structure may be provided alone or together with one or more other devices or structures. In one embodiment, one or more surfaces of a device or structure (e.g., a sample plate surface, a transfer plate surface, a channel array surface, or a reaction substrate surface may be provided with a protective layer such as a tape that can be peeled off before use. This may protect the surface from dust and other contaminants before use.

In operation, one or more devices, structures, or assemblies may be incorporated into or used with an automated solution processing device and/or signal detection device, including, but not limited to, those described herein. It should be appreciated that the orientation of the devices or structures with respect to the operator or other apparatus is not important, provided that the relative orientation of the surfaces is suited for appropriate operation. Accordingly, the terms upper and lower surfaces are used herein for convenience to indicate the relative orientation of the surfaces described.

In one aspect, through-holes connecting the inlets, outlets, transfer, exhaust and or other ports of the different structures may be of approximately the same size to assist in aligning the fluid connections between the different structures. As discussed herein, the ends of through holes may be enlarged to make registration easier. In one embodiment, a through-hole may be approximately 0.5 mm or 1 mm in diameter. However, the diameter of a through-hole may range from about 0.1 mm to about 5 mm. Of course, other diameters, including smaller or larger diameters may be used.

Applications

Methods and devices of the invention are generally applicable to any situation where a small volume of sample is added to a surface. Aspects of the invention are useful for depositing large numbers of samples on a surface, particularly when the samples are to be deposited over a small surface area. Aspects of the invention also are useful to set up a matrix of reactions by exposing lines of mobile reactants to lines of immobilized reactants on a reaction surface.

Biological Applications.

Systems and methods of the invention enable several novel approaches to multiplexing biological assays that were not available from conventional microarray or microtiter plate based approaches. These approaches can significantly reduce the amount of time and reagents required for large numbers of biological assays, thereby providing significant cost savings. In one embodiment, aspects of the invention may be used to bring one or more detection moieties into contact with one or more potential targets. This is illustrated by the examples of nucleic acid detection assays described herein. In one embodiment, aspects of the invention may be used to mix reagents for a biological or chemical reaction. For example, different reaction components (e.g. enzymes, substrates, and/or other reagents) may be mixed according to the invention. In one embodiment, one or more PCR and/or other amplification primer(s) may be mixed with substrate nucleic acid. Depending on the configuration of the assay, either the substrate nucleic acid or the primer(s) may be immobilized.

Aspects of the invention are helpful in the molecular classification of genetic diseases by providing standard testing for known molecular diseases at a relatively low cost.

Aspects of the invention also provide inexpensive multi-probe detection assays for novel unknown patient-specific molecular diseases such as micro-deletions.

Aspects of the invention also are useful in the early detection of illness, such as the detection of LOH or polyploidy in cancer. Many different tissues from the same patient or from many different patients can be tested simultaneously to increase detection sensitivity or lower cost. Rare individual cancerous cells can be detected in a field of many normal cells, and the affected tissues can be identified to enable early intervention.

Aspects of the invention can be used to simultaneously perform immunoassays for many analytes in many samples.

Aspects of the invention can be used to perform very fast hybridizations, possibly using a two channel system forming a sandwich, so that they can be used in a doctor's office to identify a target. Alternatively, fast sequential exposures of target to probe can be performed, thus enabling a few lanes to be used for many hybridization tests.

Aspects of the invention can be used for the prediction of susceptibility to disease and/or response to drugs.

Aspects of the invention can be used for scoring many SNPs (or other mutations or genetic variations) in a clinical setting. This may be useful for personalized medical treatments and/or prescriptions. In addition, a patient sample can be saved and used for subsequent genotyping with additional SNPs. Thousands to hundreds of thousands of SNPs in hundreds to thousands of individuals can be scored simultaneously. The overwhelming majority of human genetic variation is in the form of single nucleotide polymorphisms (SNPs), and it is assumed that testing for SNPs will form the basis of most genetic tests. Another significant group of genetic variations is related to the deletion or duplication of genetic sequences, which generally affect only one set of chromosomes. Deletions of sequences can be related to Loss of Heterozygosity (LOH) in cancerous cells or can be related to germ-line mutations in which one or more sequences are missing from either the maternal or paternal chromosome alleles. Duplication of genetic sequences is often found in cancerous cells.

Aspects of the invention can be used for low-cost directed sequencing for susceptibility genes like BRACA 1 and 2.

Aspects of the invention can be used for whole genome association studies of diseases and populations.

Aspects of the invention can be used for inclusive gene expression studies e.g., where the same tissue from many different patients is compared, or where many different tissue types from the same patient are compared.

Aspects of the invention can be used for sequencing, in particular for highly parallel directed or de-novo sequencing.

Aspects of the invention can be used for drug discovery. In some embodiments, highly parallel protein-protein interaction assays or drug-protein interactions assays can be performed. In some embodiments, these assays can be fluorescence polarization assays. The effect of drugs on tissue expression (e.g. gene expression) can be monitored, allowing many tissues to be tested simultaneously. In some embodiments, TaqMan assays may be used to determine drug effects on RNA expression levels.

Aspects of the invention also can be used as a general tool. For example, aspects of the invention provide a fast method of printing microarray slides with reactants such as nucleic acid targets or probes, because a large number (e.g. 384, 1536, another number of wells in a multi-well plate, or other large number over 50, preferably over 100, more preferably over 1,000, even more preferably over 10,000, even more preferably over 100,000, and even more preferably over 1,000,000) reactant spots or lines can be put down at one time on a single slide in contrast to the usual 4 to 12 for a mechanical print head.

For many applications, the management of reactant mobility may be an important feature that influences the configuration and protocol of an assay.

When an array of microfluidic channels is placed on a reaction surface to which reactants are already bound, volumes are created at the intersections of the channels with the reactants, and these volumes are similar to individual sample wells. If the results of the reactions remain local to the intersections, then these volumes act like individual sample wells. In one aspect, an advantage these pseudo-wells have over conventional assemblies of wells is that parallel rows of wells can be filled with the same reagent all at the same time, requiring minimal reagent manipulation. This may be most useful when the purpose of an assay is to expose all the individual reagents in one set to all the individual reagents in another set. For example, reactants from all of the samples stored in a 1536-well plate can be bound to a reaction surface using a microfluidic print head described herein. Similarly, all of the probes stored in another 1536-well plate can be exposed to these samples using a microfluidic reagent head described above, resulting in the maximum number of interactions between the two sets of reagents, which is 1536², or 2.3 million. One of ordinary skill will appreciate that other numbers of reactants can be mixed.

In one embodiment, when there is a sufficient quantity of reactants to perform an assay, an approach may be to immobilize one of the reactants, and perform an assays that exposes the other reactants to the immobilized reactant and may result in one or more of the other reactants binding to the immobilized reactant. This approach may be used in many hybridization assays. Sample DNA may be immobilized on a reaction surface. If one or more probes find complementary targets on the immobilized DNA and hybridize to them, the probe(s) become bound to the immobilized DNA and are thereby bound to the surface. This allows subsequent post-processing such as washing away unbound probes. This form of assay can be used to determine whether a target DNA strand is present or absent in a sample. It can also be used to determine whether the DNA strand is only present in one of the two copies of the genome (i.e. heterozygous in diploid genomes), or if there has been a duplication or other amplification of certain genes or DNA strands (e.g. trisomy). This assay can also be used to determine if there has been a single nucleotide substitution in the target strand (e.g. a SNP). Useful hybridization assays that provide increased signal to noise ratios include FRET (Roche Diagnostics), IFRET, and Molecular Beacons. Hybridization probes may be oligonucleotides. However, useful probes include any DNA, RNA, PNA, other natural, modified, or synthetic hybridization probes, and/or combinations of any two or more of the above. Probes may be from 5 nucleotides long to several kilobases long. Preferred probes include probes that are less than 10, about 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-200, 200-300, 300-400, 400-500, 500-1000, and more than 1000 bases long.

Protein-protein interactions also can be assayed by immobilization of one or more protein reactants to a reaction surface. Samples containing mixtures of proteins can be bound to the surface, the channels can carry labeled protein probes, and specific interactions may result in a probe binding to an immobilized protein target. Protein hybridization assays that can be implemented using aspects of the invention include antibody sandwich assays, and Rolling Circle Amplification tethered to a hybridization probe or antibody (Molecular Staging).

Sequencing reactions can be performed by immobilizing one or more nucleic acids to a reaction surface. For example, sample DNA molecules can be bound to the surface, and primers for specific target sequences in the sample strands can be introduced into the channels where the primers hybridize to complementary immobilized target strands. Nucleotides then may be introduced into the channels to extend the primers. Different types of primer extension reactions can be performed. For example, single base primer extension (Orchid Biosciences) can be used to genotype SNPs. In this assay, the primer may be extended by only one labeled nucleotide, which is then read to determine the genotype of the SNP. In another assay, a first nucleotide may be added and it's identity determined, then a second nucleotide may be added and it's identity determined. This may be repeated for several bases. Reagents for such assays are available, for example, from Pyrosequencing. Photocleavable fluorescent nucleotides or dideoxynucleotides developed at Columbia University also may be used.

Highly-parallel sequencing of novel DNA strands also can be performed using immobilized DNA molecules. According to the invention, there are at least two possible approaches. The first uses random primers in the channels to initiate sequencing reactions. The second involves sequentially introducing random labeled primers into the channels, recording those that hybridize, then assembling the results into a representative sequence. In both embodiments, sample DNA may be prepared carefully so that not more than one probe hybridizes to the sample DNA at each intersection of bound DNA and probe DNA. A useful sample preparation method accomplishes this by processing each sample so that it consists of many copies of relatively short DNA strands.

These and other applications are discussed in more detail below.

Nucleic Acid Hybridization Assays:

As discussed above, aspects of the invention may provide efficient methods for multiplexing hybridization assays involving crossing sample lines and probe lines.

In one embodiment, each sample may be deposited as a single vertical line on a slide and each probe may be introduced as a single horizontal line. Each vertical line of sample may interact with all of the horizontal lines of probe, thus providing an assay platform where each intersection of sample and probe represents a unique data point. Therefore the number of tests performed on each sample (held in a single well and introduced as a single vertical line) may be equal to the number of horizontal lines of probes introduced to the device. If 1536 probes are used, then the level of sample multiplexing is 1536 tests per sample well. Conventional microtiter plate based assays are capable of only from one to two tests per sample.

When performing a hybridization assay of the invention, the wells of a microtiter plate may be fluidically connected to a set of parallel channels of a microfluidic device through a transfer plate, as described above. The array of microfluidic channels may be mounted on a reaction substrate that communicates with all the channels and is capable of binding DNA that is introduced into the channels. A DNA sample may be introduced at a sufficiently high concentration to ensure that all the different targets in the sample will be represented at every reaction site formed by the intersection of bound DNA lines and mobile probe lines. When genomic samples are used, they are usually amplified using a whole-genome-amplification method and then sheared or cut using restriction enzymes to produce short strands on the order of 1000 nucleotides long. Other shearing methods also can be used. The short strands then may be randomly mixed so that there are enough copies of all short strands at every intersection point to enable their detection. Current Whole Genome Amplification kits can make approximately one million copies of a genome. Therefore, if 1000 channels cross a line of this amplified sample DNA, approximately 1000 targets may be available at each intersection point. This number of targets can be detected using currently available instrumentation and probe labels. Further increases in the number of genome copies or the use of more sensitive instrumentation that can detect the presence of fewer labels will enable more probes to be exposed simultaneously to each line of amplified DNA.

After lines of sample DNA are deposited on a reaction surface, the first array of microfluidic channels may be removed. The bound DNA may be immobilized, preferably using a UV oven. The reaction surface then may be blocked to prevent non-specific binding of labeled probes. A second array of microfluidic channels may be contacted to the surface, with the channels at an angle (e.g., a 90 degree angle) relative to the sample lines. Labeled DNA probes specific for targets within the sample DNA then may be introduced into microtiter plate wells and directed to the microfluidic channels where they are exposed to the immobilized DNA samples on the reaction surface. Each channel may contain a single type of labeled DNA probe. At the intersections of the immobilized DNA lines with the channels filled with probes, labeled probes hybridize with complementary targets that may be present in the sample DNA. The probes may be left to hybridize for between 5 minutes and 12 hours depending on the reaction conditions and the probe and target concentrations. However, shorter or longer hybridization times may be used. Typical reaction conditions may be used, following standard hybridization protocols used in microarray hybridization reactions. In another embodiment, one or more probe solutions may be flowed across one or more lines of bound nucleic acid without stopping the flow(s) for any length of time. Sufficient hybridization may occur in the time that it takes for a volume of probe solution to move across a region of bound target.

After hybridization is complete, the second array of microchannels may be removed from the reaction surface, and the reaction surface may be washed to remove any unhybridized probes. The surface then may be examined for the presence of any remaining label that would indicate that hybridization took place.

In another embodiment, one or more probes may be deposited and immobilized on a reaction surface. One or more nucleic acid samples may be labeled, flowed over the immobilized probes, and washed off. Any hybridized nucleic acid remains bound to the immobilized probe and may be detected.

In either configuration (bound target and mobile probe, or bound probe and mobile sample), the matrix of hybridization reactions may have several advantages over hybridization reactions performed with available nucleic acid microarrays. In some embodiments, advantages may result from the small volume of each hybridization reaction as discussed in the following paragraphs. According to aspects of the invention, small reaction volumes and resulting short diffusion distances are advantageous not only for hybridization reactions, but also for many other embodiments of the invention.

Increased Accuracy and Speed

Hybridization assays depend on the ability of DNA in solution to migrate to the site of immobilized DNA on the reaction surface to find a hybridization match. In current microarray and bead-based assays, DNA molecules several thousand bases long must migrate up to two centimeters to hybridize with oligonucleotides that are attached to flat surfaces, and are only 25 or so nucleotides long. Because of the slow diffusion coefficients and the difficulty to significantly agitate current samples during hybridization, the minimum time needed for hybridization on microarrays is about 12 hours. Easier agitation methods using bead-based assays make hybridization times much shorter. However, even during these times, it is probable that only a small number of potential targets are exposed to the probes attached to the microarray surface, resulting in less efficient hybridization and poor signal-to-noise ratios. Significant steric hindrance also exists in both microarray and bead-based assays due to the long target having to approach a flat surface for hybridization, resulting in reduced hybridization stability differences between matched and mismatched target-probe combinations. Both of these effects result in the requirement of duplicate hybridizations per target, to increase the confidence levels of the observed results.

In contrast, in some embodiments of the invention, probes may be only 18 to 30 nucleotides long and migrate at most 50 microns, or the width of a microfluidic channel, to their hybridization sites. Also probes typically have much larger diffusivities than the larger sample DNA. As a result, hybridization reactions of the invention may be faster and may result in a greater percentage of probes being exposed to potential hybridization sites, resulting in increased hybridization efficiency. Furthermore, the steric hindrance of a short probe approaching a wall may be smaller than that of a large DNA fragment, resulting in increased hybridization accuracy. Overall, these effects may result in much greater hybridization efficiency and fidelity thereby reducing or eliminating the need for duplicate hybridization sites.

Increased Scalability

The size of the hybridization area of conventional microarray slides is generally not larger than 2 cm² because of the difficulty in transporting the long-chain DNA targets to the immobilized probes. Arrays provided by, for example, Affymetrix may be no larger than about 1.4 cm², containing about 1.3 million probe spots. Labeled targets must cross this whole surface area to become exposed to probes that may be complementary.

In contrast, in some embodiments of the invention probes may brought to within 10 to 50 microns of potential complementary targets by active fluid flow through the channels. Therefore, in aspects of the invention, the size of the hybridization area is not limited by the distance that DNA strands can diffuse or be moved by gentle agitation, but may be limited only by the practical length of channels that can be made in an assembly of the invention. For example, a channel that is 18 centimeters long may be made and would provide a total hybridization area of 324 square centimeters, containing 160 million spots, or 100 times more spots than a slide provided by Affymetrix. Given that an Affymetrix slide needs 20 duplicate spots for each hybridization event, devices of the invention may score 2000 times more hybridization events that an Affymetrix slide. If the 160 million spots were used for de-novo sequencing by methods discussed herein, each strand would only theoretically need to be read out to about 140 nucleotides to sequence the entire human genome.

Reduction of Sample Preparation and Amplification Cost and Complexity

Current microarray and bead-based assays use selective amplification of DNA strands containing targets of interest from an initial genomic sample. This is necessary in order to label the target strands and amplify the number of labeled strands for easier detection of the labels. PCR primers must be optimized for each target, and only a few targets can be amplified per sample well, requiring manipulation of many microtiter plates and expensive PCR reagents. In addition, increased numbers of targets requires increased quantities of patient sample DNA which is consumed during the target amplification and labeling process.

Techniques available from Affymetrix and Perlegen may allow cheaper methods to amplify and label targets by ligating universal primers to genome fragments and causing selected areas of the genome to be amplified sufficiently to be used with a microarray slide that is spotted with short oligomer probes. This approach allows the user to genotype approximately 10,000 targets in a sample.

A genome amplification technique introduced by both Amersham and Molecular Probe Inc is called Whole Genome Amplification. This method uses a novel enzyme to amplify the whole genome from about 200 copies to about one million copies. This method does not label the amplified DNA, and is therefore not appropriate for current hybridization assays. However, this method can be used to prepare sample DNA for analysis according to the invention. This avoids the costs associated with PCR primers and the use of multiple sample wells for PCR reactions. Other aspects of the invention described herein also are useful for simplifying sample processing and analysis.

Multiplexing by Blocking

In embodiments described herein, each vertical line may consist of a single sample of, for example, the full diploid genome of a certain species. This may result in the ability to reach a high level of sample multiplexing. However, in some embodiments, only one test type may be performed per horizontal channel, or per probe. In other embodiments, it may be desirable to increase the number of tests per horizontal channel. This can be achieved by making available only a portion of a single sample in a single vertical line. For example, a full genome could be broken into ten unique parts, and each part could be introduced into a vertical line. Then each horizontal line could carry ten sets of probes, where each set of probes only had targets on one of the ten lines of sample. This would result in multiplexing of the horizontal lines in that ten tests would be performed per horizontal line, or probe. Therefore, the level of multiplexing per vertical line or sample portion would still be equal to the number of horizontal lines carrying probes. However, the total level of multiplexing per total sample would be equal to ten times the number of horizontal lines carrying probes.

It is generally very difficult to break up a sample into a number of unique parts, in that the DNA in the sample is all well mixed together. However, in aspects of the invention this effect can be achieved by using blocking probes as follows. The same genomic sample may be introduced into each of ten vertical lines. Then unlabeled probes may be introduced into each of the lines, where the probes in each line hybridize to and block all target sites in the assay except for the target sites that are intended to be probed in that line. Then all the probes for all the vertical lines are mixed together in the horizontal lines. At each intersection of line and probe, only the targets that have not been blocked will be available for hybridization. This achieves the effect of having each vertical line consist of a unique segment of the genome being tested.

In an alternative embodiment, the reaction surface first may be exposed to the lines of target DNA. Then a second channel device may be used where each line contains not a single labeled probe but instead contains all the (non-labeled) probes except for the probe of interest for purposes of blocking. As a result, all the target spots except for the target of interest may be blocked. Then the channel device may be removed and the whole slide may be exposed to a mixture of all the (labeled) probes for all the targets of interest. The labeled probes hybridize to spots where single targets are open. The resulting hybridization pattern can be used to determine the presence or absence of the target. Other aspects of the invention involve using other combinations of blocking and detection probes.

PCR Multiplexing

In one embodiment of the invention, samples may be introduced as vertical lines on a slide and PCR primers may be introduced as horizontal lines. Each sample may contain all the forward primers for the intended amplicons from that sample. Reverse primers may be introduced into the horizontal channels such that at each intersection of sample and primer there may be only one matched pair of forward and reverse primer. During PCR amplification, all (or substantially all) the forward and reverse primers present at each intersection of sample and primers may extend linearly. However, the unique set of forward and reverse primers at each intersection will amplify exponentially, generating orders of magnitude more of the intended single amplicon than of the linearly-amplified amplicons from the unmatched primers. This will result in exponential amplification of unique amplicons at every intersection of bound sample DNA and channel. Therefore the number of PCR amplifications performed on a sample is again equal to the number of reverse primers that are introduced to the device. If 1536 primer sets are used and 1536 samples are used then over 2 million PCR reactions can be assayed. In contrast, only 20 or so PCR amplifications can generally be performed on a single sample in a single well, because primer dimer formation becomes a major obstacle when more PCR amplifications are attempted.

Non-Immobilized Reactants

In some embodiments of the invention, members of a first set of reactants may be exposed to members of a second set of reactants without immobilizing either set of reactants on a surface. However, the reactants still may be constrained by microfluidic channels, and the interaction points still may be defined by the intersection of lines of microfluidic sample flows.

For example, when there is not a sufficient concentration of DNA to perform an assay, PCR can be used to amplify the nucleic acid strands of interest. The new DNA strands produced are not immobilized and may migrate along the channels. This may require other methods to contain the reactants within the pseudo-wells. One method is to rely on the diffusivity of the reactants. If they have sufficiently low diffusion coefficients, they will diffuse slowly enough to remain in the pseudo-wells over the time-course of the reaction and/or analysis. DNA strands that are kilobases long will diffuse out of a 10-micron well in about 1.3 hours, and out of a 100 micron well in about 130 hours. DNA strands that are 1 Kb long will diffuse out of a 10-micron well in about 4 minutes and out of a 100 micron well in about 7 hours. DNA strands less than 25 bases long will diffuse out of a 10-micron well in about 6 seconds, and out of a 100 micron well in about 10 minutes. Therefore, only longer-chain reactions may be practical when reactants are not immobilized or prevented from diffusing into adjacent wells.

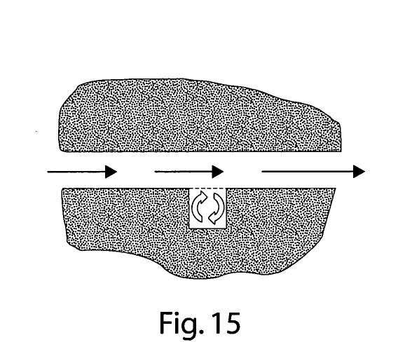

According to aspects of the invention, a useful device configuration for when diffusion is relied upon to retain reactants is shown in FIG. 15, where the reaction surface is replaced with one or more microfluidic channels. As a result, two sets of channels cross each other at an angle (preferably a right angle) and communicate with each other.

According to aspects of the invention, simple hybridization reactions can also be performed with the configuration shown in FIG. 15. One set of channels is loaded with long-chain DNA samples that diffuse at a rate that is slow enough that they can be considered as immobile. The other set of channels is loaded with labeled probes, which diffuse at a higher rate and hybridize to the long-chain target DNA. Excess-labeled probes are then washed out of the second set of channel and the remaining labeled primers are detected.

In another embodiment, the reactants may be contained within the pseudo-wells by supplying the channels with structures that can physically isolate channel volumes at their intersections with the immobilized lines of reactants. These structures open to allow entry of reactants to the whole channel, then close to isolate the channel volumes. One approach to isolating reactants along a channel is shown in FIG. 16. Vertical dams, or other structures can be placed within the channels such that when pressure is applied to the tops of the channels, they are partially or fully obstructed, so as to prevent either fluid flow or diffusion of reactants along the length of the channels. This allows the creation of sections along the channel that can act like individual isolated sample wells.

In another embodiment, reactants can be contained within the pseudo-wells by providing a means for reactants to become attached to the walls of the reaction surface or channel during or after a reaction. One approach that can accomplish this may be to bind primers to the reaction surface and introduce long-strand, slow-diffusing, template into the channels. The template nucleic acid first may be broken up using one or more restriction enzymes, and the fragments may be linked to universal primers. The template may be introduced at a low concentration so that at most there may be one nucleic acid fragment at each channel intersection. An immobilized primer that hybridizes to a universal primer on a nucleic acid fragment may be extended. Every extension product made from the template also may become bound to the reaction surface. This approach can be particularly useful when the intent is to amplify up only one copy of template DNA to produce a large quantity of amplicons that are all copies of the single DNA strand. Amplification from single strands of DNA is sometimes referred to as “digital PCR”, and is useful for detecting haplotype genetic variations and for detecting individual mutated cells in a field of many normal cells for early stage cancer detection. Once the single-copy amplicons are generated at the intersection of the channels and bound primers, then these can be exposed to probes or sequenced according to the methods presented herein.

Whatever protocols are used to restrict the results of reactions from migrating, aspects of the invention provide the ability to perform highly-multiplexed PCR reactions. Sample DNA can be introduced into vertical channels, where each sample also contains all the forward primers for the intended amplicons from that sample. Reverse primers can be introduced into the horizontal channels, where each channel contains a unique subset of all the reverse primers for the intended amplicons for every sample. This can result in exponential amplification of unique amplicons at every intersection of bound sample DNA and channel. Examples of assays that could be used with this approach are allele-specific PCR, to both amplify and identify alleles, or a quantitative PCR TaqMan assay.

Combinations with Other Array Technologies

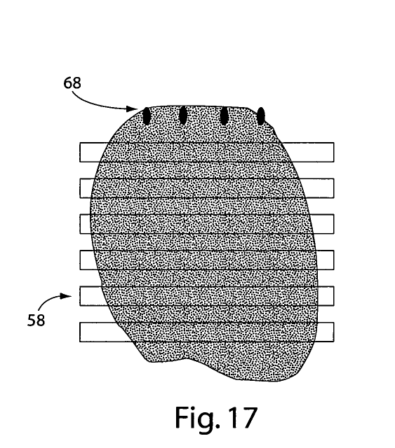

According to aspects of the invention, additional assay configurations can be obtained using a reaction surface constructed by standard microarray techniques, where each spot on the microarray represents a unique strand of DNA, as shown in FIG. 17. Alternatively, the DNA can be attached to a reaction surface as a series of spots using microarray methods of the invention. For example, a microfluidic channel in the microfluidic array can be open for a very short distance, thereby contacting the reaction surface for only a very short distance. A microfluidic array comprising a plurality of such microfluidic channels can be used to deposit a matrix of reactants such as probes or targets on a reaction surface. In some embodiments, a microfluidic channel could contact the reaction surface at several discrete positions in order to deposit duplicate (or triplicate, or more) samples on the reaction surface. The surface can then be covered with an array device of the invention to expose one or more labeled probe DNA reactants to the immobilized DNA samples.

In other aspects of the invention, similar configurations can be used for other types of nucleic acid reactants (e.g., RNA or PNA), non-nucleic acid reactants (e.g., peptides, proteins, carbohydrates, small molecules), or combinations of two or more thereof.

High Density Arrays

Microfluidic embodiments of the invention enable the number of hybridization spots per unit area on a microarray reaction surface to be greater than can be achieved using a spotting approach, and to meet or exceed the number obtainable with lithographic techniques. The number of hybridization spots per area may be maximized so as to produce a maximum number of test events per assay protocol. Conventional physical spotting techniques such as quills, pins, or micropipettors are able to deposit DNA on glass slides in the range of 60 to 250 microns in diameter, resulting in from 400 to 7000 spots per square centimeter, allowing for clearance between spots. Lithographic techniques, such as those provided by Affymetrix (e.g., U.S. Pat. No. 5,744,305, the disclosure of which is incorporated by reference herein) can produce hybridization spots down to about 11 microns square, with no clearance between spots, resulting in approximately 800,000 spots per square centimeter. Since the quantity of probes that can be deposited on a flat surface using lithographic techniques is small relative to the quantity achieved using mechanical spotting techniques, approximately 20 duplicate spots may be required for each target. This reduces the number of targets that can be probed on a standard lithographic array to about 40,000 per square centimeter.

According to embodiments of the invention, microfluidic technology has been used to make 50-micron square hybridization spots with 50-micron separators between channels, resulting in 10,000 spots per square centimeter. Microfluidic channels also have been made as small as 10 microns, with 5-micron spacers between channels. Therefore, a device of the invention can include over 400,000 spots per square centimeter. These spots contain much more DNA than the spots on a standard lighographic array. Therefore, fewer duplicate spots are needed. If, for example, no duplicates are used, then microfluidic arrays of the invention can exceed the density of spots achievable by standard lithography by a factor of 10 or more.

In one aspect of the invention, an advantage of using channel widths and heights of 10 microns is that each of the 400,000 wells on a square centimeter has an approximate well volume of 1 picoliter. However, aspects of the invention include other channel heights and widths as described herein.

Kits

In addition to the devices described above, aspects of the invention provide kits that contain preassembled components or reagents that can be readily used in conjunction with methods and devices of the invention.

Pre-Packaging of Probes with an Array

Probes that are intended to be exposed to a reactant (e.g. a sample) can be preloaded into a microfluidic chip containing a microfluidic array as described herein, and may be sealed. A microfluidic chip may consist of only a soft chip part, and may not include a microtiter plate or transfer system. There may be a number of reservoirs in the chip, each connected to the channels on the bottom of the chip. The chip could carry any number of channels and reservoirs, and would not be limited by the number of wells that are in standard microtiter plates. For example, the chip could have 2000 different reservoirs and channels, rather than 1536, which is the number of wells in a 1536 well plate. The configuration of the chip may then be made so as to fit the configuration of the surface that will hold the targets, rather than be restricted by the number of wells of a microtiter plate. After the reservoirs are loaded, the microfluidic device may be packaged into an air-tight film bag to keep the probe samples fluid. To use, a user may remove the chip from the package, then place the chip onto the slide where target DNA has been already deposited. Then the chip may be pressurized (e.g., with either positive or negative pressure) to drive the probes from their reservoir into the channels and thus expose them to the target DNA. In another embodiment, probe may be dried down in the array (e.g., upstream from a channel portion that may contact a reaction surface). The operator may then flow the probe over the reaction area by flowing a reaction solution over the dried probe region in each channel.

This aspect of the invention provides several benefits. One is that it unburdens the user from needing to secure and array the probes into a microtiter plate. It also relieves the user from needing to apply a microtiter plate-microfluidic chip combination to a glass slide and correctly aligning all the device components. Instead the user would just apply the small chip to the surface of the glass slide, and apply pressure to the chip to force the probes from their reservoirs to the channels and across the areas of immobilized reactant (e.g., target DNA). Another benefit is that it provides a way for a vendor to pre-package a set of probes for a customer. Such a device may be a one-time consumable.

Pre-Packaging of a Reaction Surface with a with a Channel Array

In some aspects of the invention, a channel array also may be pre-positioned onto a reaction surface (e.g., reversibly bound to a reaction surface), such that it is ready to receive a reactant solution (e.g., target DNA). In operation, reactant solution could be dispensed directly through the channel array either manually or using an automated pipettor. Alternatively, the pre-positioned array and reaction substrate could be fitted together with one or more of a multi-well plate, a transfer plate, a docking interface, and/or any other device of the invention.

In embodiment, the pre-positioned reaction substrate includes one or more reactants (e.g., hybridization probes) on its surface. The reaction substrate may be an array (e.g., a micro-array) of reactants made using any method (including methods of the invention). For example, the reaction substrate may be a micro-array available from a commercial source (e.g. Affymetrix, Agilant Technologies, GE Life Sciences, Perkin Elmer, etc.).

This aspect of the invention may provide several benefits. A user could interrogate a pre-packaged assembly by flowing a mobile reactant solution (e.g., a sample suspected to contain target DNA) over the reaction surface. The array then may be removed from the surface of the slide and analyzed for the presence of a signal of interest that may be indicative of the presence of a particular target of interest in the sample.

Pre-Packaging Target DNA Loading Probes and a Reaction Surface to Hold DNA Together

In aspects of the invention, a chip also may be designed to combine channels for depositing target DNA onto a glass slide and also exposing probes to the immobilized target DNA. The chip would have channels running both vertically and horizontally on one side, where the two sets of channels intersect. Therefore the bottom of the microfluidic device would appear as a series of squares, separated by channels running either vertically or horizontally. One of the ends of one set of channels would be connected to reservoirs that held probes, and the other ends of these channels would be dead-ended. One of the ends of the second set of channels may be connected to ports on the surface of the device that enabled them to be filled with target DNA either manually or robotically, or by fitting a microtiter plate and transfer layer to the device. The other ends of these channels would connect to a single common exhaust port.

In order to use the device, target DNA would be introduced into the proper channels, and vacuum would be placed onto the common outlet to these channels. The target DNA would be drawn into these channels, where it would bind to the reaction surface. This target DNA would not enter the other orthogonal (target) channels, because the DNA would be under vacuum and because one end of the orthogonal channels would be dead-ended, and the other end would connect with reservoirs which were filled with probes, and sealed. After the target DNA was introduced into the device and removed, by means of vacuum, then the reservoirs of probe DNA would be pressurized to force the probes down their respective channels where they would cross the bound DNA channels. The pressure on all the probe DNA channels would be kept equal so as to minimize any tendency for probe DNA to cross from channel to another by means of the cross-target DNA deposition channels. After a suitable hybridization time, the microfluidic chip would be peeled off of the flat surface or glass slide, and the slide would be washed, then analyzed for places where hybridization had taken place. It should be apparent that other inlet, outlet and channel configurations can be used for this aspect of the invention. Also, this aspect of the invention is not limited by the reactants that are used.

### EXAMPLES

### Example 1

### Parallel Lines of DNA on a Glass Slide

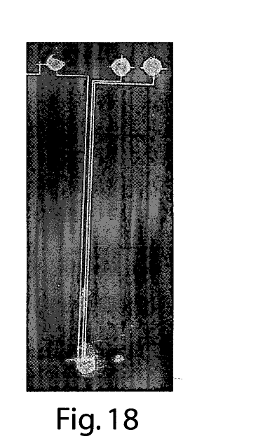

FIG. 18 shows an embodiment where an array of microchannels was used to immobilize DNA into parallel lines on a glass slide. The round spots at the top of the image are wells or inlets where individual DNA samples were introduced to the array of microchannels. These wells are fluidically attached to channels that direct the DNA along the glass slide, where it is immobilized. The array of microchannels was then removed, and the slide was exposed to cyber green dye to make the lines of DNA visible. While these lines are approximately 50 microns in width, in other embodiments, they may be as small as 10 microns in width or smaller.

### Example 2

### Matrix of Hybridization Reactions

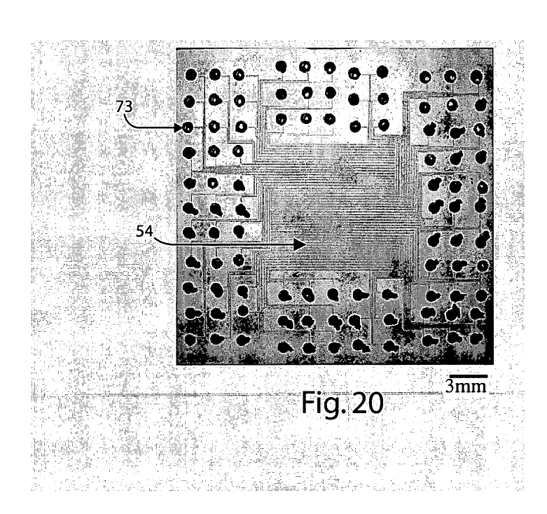

FIG. 20 shows a micrograph of a 96-channel microfluidic device that was used in the experiments described below. Fluid inlet ports 73 are shown (these ports are through holes that are in communication with the upper surface). Each microchannel 54 is 50 microns wide (these are on the lower surface). The device was first placed on a glass slide with the channels oriented vertically. Sample DNA was then allowed to flow through a selected number of channels for less than a minute before it was removed from the channels. The device then was removed from the slide. The slide then was treated to bond the sample DNA to the glass slide, followed by blocking to prevent any other DNA from adhering to it. The same microfluidic device was again applied to the glass slide with the channels oriented horizontally. Selected channels were then filled with labeled probe DNA, and the assembly was allowed to incubate for 12 hours. Subsequently, the microfluidic device was removed from the glass slide, which then was washed to remove any unhybridized probe DNA. A fluorescence image of the slide was then taken to show the positions of the hybridized labeled probe DNA.

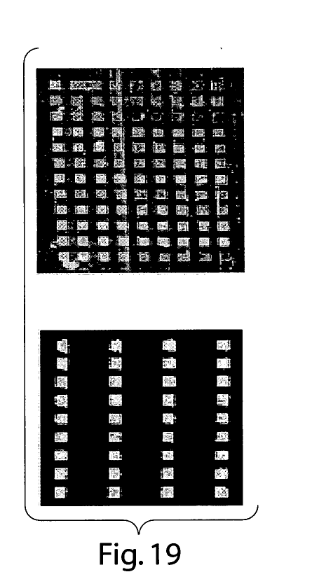

FIG. 19 shows the results of experiments that demonstrate the ability of an array of microchannels to hybridize labeled DNA probes to lines of DNA previously immobilized on a glass slide, and for the probes to discriminate between two different targets. In the upper image, a first array of microchannels was first used to immobilize vertical lines of DNA (Beta Actin) on a glass slide. In this first array, each microchannel was 50 microns wide. Then a second array was placed on the glass slide with the microchannels in a normal orientation relative to the orientation of the DNA lines deposited by the first microchannels. The microchannels in the second array were also 50 microns wide. This second array was used to expose 50 micron wide horizontal lines of fluorescently-labeled complimentary DNA (Cy-3 labeled UHR). The labeled DNA was left exposed to the immobilized DNA for 12 hours. The array then was removed and the glass slide was washed to remove any unhybridized DNA. This resulted in the appearance of squares of labeled DNA where the channels crossed the lines of immobilized DNA.

In the lower image of FIG. 19, alternating vertical lines of human DNA and Drosophila DNA were deposited on the glass slide. Then horizontal lines of cy5-labeled probe specific for Drosophila were exposed to the targets, resulting in hybridization to only the Drosophila target, thus uniquely identifying the presence of this target.

### Example 3

### Reuse of Patient Samples for New Targets

The arrays of the invention allow for sequential assays to be performed on sample DNA that has been attached to a reaction surface. Reuse of sample DNA provides two significant advantages: a) the cost of sample preparation can be spread over many uses, and b) the sample can be probed for new targets that were not contemplated when the DNA sample was originally prepared. According to the invention, samples deposited on a reaction surface can be conserved for tests to be performed at a future date.

In preferred embodiments of the invention, whole genome amplifications are performed and the resulting DNA samples are deposited on the reaction surface. As a result, all targets contained in the genome are potentially available. Therefore, once the DNA on the reaction surface has been exposed to channels of a first set of hybridization probes, other targets are still available for future hybridization assays.

Once a hybridization assay is completed, the labels on the probes can be neutralized, and a second set of probes can be exposed to the sample DNA. It is expected that any steric hindrance from the presence of a first set of probes will be minimal, allowing successive exposure of probe sets to the sample DNA. Alternatively the probes can be removed from the sample DNA by strong washing of the reaction surface. Both approaches have been used to reuse porous membranes that have been used in Dot Blots. In either case, as long as the sample DNA is firmly attached to the reaction surface, there should be no limit to the number of times the immobilized DNA can be exposed to newly labeled hybridization probes.

In contrast, labeled amplified targets used by current microarray and bead-based assays cannot be reconstituted and reused for sequential assays, and also cannot be used to probe for additional targets that were not originally amplified and labeled.

### Example 4

One of the goals of the National Human Genome Research Institute, (NHGRI) is to assay 400,000 SNPs in each of 2000 different patient samples. One way to accomplish this using microchannel methods and devices of the invention would be to cross 2000 vertical sample lines and 400,000 horizontal probe lines. If the distance between each line were 15 microns, the microfluidic chip would be 3 centimeters wide by 600 centimeters long. The number of microtiter wells needed to hold this number of samples and probes would be 402,000 (e.g. 262 microtiter plates each holding 1536 wells). This approach would therefore require a) an unwieldy microchannel chip shape, and b) many manipulations to transfer samples and probes from many microtiter plates.

In a preferred embodiment, each sample would be divided into 14 unique parts of the whole genome, using blocking probes on the chip as discussed previously. The horizontal lines would each contain 14 different probe sets, or the full complement needed to probe all 14 segments of the genome. The resulting chip would have 14×2000=28000 vertical sample lines and 400,000/14=28,600 horizontal lines, resulting in a chip that was 42 centimeters by 42 centimeters. The number of microtiter wells needed to hold samples and probe mixtures would be only 30,600. This number of wells could be provided by 20 microtiter plates each holding 1536 wells. Therefore, this approach would result in a more user-friendly chip size and a dramatically reduced number of microtiter plates to set up and manipulate.

Similar results may be obtained using other embodiments of the invention.

### Example 5

### Disease Detection

Genetic testing can be used for accurate molecular classification of disease, early detection of illness, prediction of drug response, and prediction of susceptibility to disease. There are over 2000 known genetic polymorphisms that lead to disease states that are currently not available as tests, because of the high cost of the assays. Commercial companies and most medical centers also perform cytogenetic tests that involve a visual examination of a patient's chromosomes under a microscope. This is an inexpensive way to detect gross abnormalities such as extra or broken chromosomes. Such tests are often performed on a prenatal basis. However, a significant portion of chromosomal abnormalities, such as deletions and rearrangements, cannot be detected visually and require advanced molecular techniques for detection. Even though these types of abnormalities are relatively common and almost always very serious, there are no publicly available diagnostic tests, because they would be too expensive since they cannot be automated on an inexpensive platform.

Genetic testing can be used for the early detection of different types of illness. First, pathogens causing conditions ranging from the common cold to more serious conditions such as hepatitis can be detected and identified through genetic testing. Second, genetic testing is useful to detect many forms of cancer at an early enough stage for successful treatment. Two issues complicate molecular-based detection of cancer. The first issue is finding the few cancer cells in a population of many normal cells, since they generally remain local to the affected site. The second issue is the molecular detection of a suspected cell, because the type of genetic disruption and the exact position on the chromosomes of an affected cell varies widely with the type of cancer and the individual patient. Therefore, successful molecular detection may involve a combination of (a) testing many cells to detect a small percentage of cells that may be cancerous, and (b) the use of many diagnostic markers to cover the wide range of genetic disruptions that may be present in cancerous cells. Because of the high cost of performing molecular detection tests, very few tests are available either for research or clinical purposes, even though the benefits to early stage cancer are well known. Aspects of the invention may be used for performing many reactions simultaneously, and may reduce the cost and complexity of handling many samples along with many reagents.

### Example 6

### Analysis of Drug Responses

The use of genetic testing for the prediction of drug response has a high potential for increasing the effectiveness and reducing the side effects of drug therapy. Additionally, genetic testing holds promise for dramatically reducing the costs of drug development. Commercial drugs generally only work well for a percentage of the population (can be as low as 30%). The same drugs can have no effect on other significant portions of the population, and can also result in serious side effects. Also, there is evidence that genetic polymorphisms either cause or increase the susceptibility of patients to drug response. Genetic testing may, therefore, become a significant part of drug development and therapy. However, genetic testing is currently far more expensive than most drug prescriptions. Until test costs come down to the level of drug prescriptions, they may only be used in critical situations, even though general use could greatly increase the effectiveness of many drug therapies. Aspects of the invention may be used for drug screening and evaluation, and may reduce the cost and complexity of drug assays.

### Example 7

### Protocol for Using PDMS Chips to Print and Hybridize DNA Microarray Slides, where Small Labeled Oligonucleotides are Hybridized to Printed Genomic DNA

The following non-limiting example illustrates operational aspects of the invention. The illustrated protocols may be used alone or in combination. In one embodiment, the hybridization step may be performed on a pre-printed slide as described herein.

According to one method of preparing a PDMS chip, the chip is first washed with soap and tap water, and it's channels are scrubbed with soft sponge. Then, the chip is immersed in a sonicating bath of 2×SSC, 0.1% SDS for 5 minutes to remove bound protein from earlier uses. The chip is then rinsed in H2O, IPA, and then dried, such as by air drying.

In one method, glass slides may be used as reaction substrates without any preconditioning. Corning Gaps II slides are an example of such slides.

In one method, preparations for printing DNA include preparing printDNA Concentrations between 200 to 800 nM, and preparing a printing buffer of 3×SSC. Then, for each sample to be printed, about 5 μl DNA is prepared. If the DNA is double-stranded, the samples are denatured before printing by heating them to 95° C. for 10 min, placed in ice for 5 minutes, and then spun briefly to recombine any condensate with the sample.

Preparation of a Labeled DNA to be Hybridized:

In one method for preparing labeled DNA to be hybridized, concentrations should be more than 10 pM to be positively detected. For each sample to be hybridized, 6 μl of sample volume are prepare. The amounts shown in Table 1 are then used when 10 pM of DNA is to be hybridized. For different concentrations, the initial dilution of the DNA sample may be adjusted accordingly:

TABLE 1

| | Final | Volume, |
|---|---|---|
| Hybridization Buffer and sample | conc. | μl |
| DNA Sample (pre dilute to 60 pM before | 10 pM | 1 |
| adding) | | |
| Formamid (100%) | 25% | 1.5 |
| SSC (at 20x) | 5X | 1.5 |
| SDS (at 10%) (pre-dilute to 0.6% before | 0.10% | 1 |
| adding) | | |
| Salmon Sperm (or BSA or herring sperm) | | |
| (pre-dilute to 6 mg/ml before adding) | 0.10% | 1 |
| Total | | 6 |

According to one illustrative method, for each hybridization chip, an additional 6 μl of blank hybridization buffer may be prepared, substituting DEPC water for the DNA sample. If the labeled DNA is double stranded, immediately before hybridization, the samples may be denatured by heating them to 95° C. for 10 min, then place in ice for 5 minutes, then spin briefly to recombine any condensate with the sample.

In one example of printing a slide, the PDMS chip is placed onto the surface of the glass slide, and then examine the slide to determine whether debris is blocking the channels. The slide may then be labeled to mark the area containing the channels 500 nl of DNA sample is then loaded onto each chip channel entrance. 3 inches hg vacuum are then applied to the channel outlet to move the samples through the chip in approximately 5 minutes. Optionally, each channel is then rinsed with 400 nL 3×SSC. The chip may then be removed from the slide while vacuum is still being applied, taking care to avoid splashing excess liquid across the slide.

For post printing, in one optional example, the slide is treated by either UV Cross-linking the slide at 65 mJ or baking the slide to fix the DNA onto the slide surface. The slide is then dried in room air, typically for between 10-15 minutes. Afterwards, the slide is stored at room temp, away from light.

For hybridization in one embodiment, the PDMS chip is placed onto the surface of the glass slide so that the channels cross the lines of printed DNA, and then the slide is examined to determine whether debris is blocking the channels. Then, the slide is placed on a heating block at from 38 to 42° C. 500 nl of DNA sample are loaded onto each chip channel entrance. Using 3 inches hg vacuum on the channel outlet, the samples are through the chip in approximately 5 minutes. Subsequently, the each channel may be rinsed with 400 nL of blank hybridization buffer. The chip may then be removed from the slide while vacuum is still being applied, taking care to avoid splashing excess liquid across the slide.

To complete a stringency wash in one example, the array is immersed array in 2×SSC, 0.1% SDS for 5 minutes at 42° C. in a 50 ml conical tube, inverting tube once each minute. Then, the array is transferred to 0.1×SSC, 0.1% SDS for 10 minutes at 42° C., inverting tube once each minute. Subsequently, the array is transferred to a new container of 0.1×SSC, 0.1% SDS for 5 minutes at RT, inverting tube once each minute. The array is then quickly rinsed with 0.1×SSC for 5-8 seconds and dried using clean compressed air or nitrogen, or in centrifuge at 1600 g for 2 minutes.

1. A method of forming a line of sample on a surface, the method comprising the steps of:
(a) forming a contact between a reaction surface on a reaction substrate and an open microfluidic channel on a channel substrate;
(b) introducing a sample solution into the microfluidic channel, wherein the sample solution contacts the reaction surface along a contact line formed by the contact between the reaction surface and the open microfluidic channel; and,
(c) disrupting the contact between the reaction surface and the microfluidic channel, thereby forming a line of sample on the reaction surface, wherein the line corresponds to the contact line.

2-4. (canceled)

5. The method of claim 1, wherein the contact line of the microfluidic channel is between 1 micron and 500 microns wide.

6. The method of claim 1, wherein the contact line of the microfluidic channel is between 1 um and 500 um deep.

7-11. (canceled)

12. The method of claim 1, wherein the channel substrate comprises a plurality of open microfluidic channels, and a plurality of sample solutions are introduced into the microfluidic channels.

13-15. (canceled)

16. The method of claim 12, wherein the microfluidic channels are separated by a plurality of channel walls each having a thickness of between 5 and 50 microns.

17. The method of claim 12, wherein said plurality consists of between 10 and 2000 microfluidic channels.

18-34. (canceled)

35. A reaction surface array comprising a plurality of lines of biological samples, wherein the array is produced according to the method of claim 1.

36. A reaction surface array comprising a plurality of lines of reagent, wherein the array is produced according to the method of claim 1.

37. The surface array of claim 35, wherein the array comprises about 2000 lines of biological samples.

38. The surface array of claim 36, wherein the array comprises between 10 and 400,000 lines of reagents.

39. An array of target samples with a density of at least 50 sample lines per linear centimeter.

40. A method of contacting each member of a first plurality of samples with each member of a second plurality of samples, the method comprising the steps of:
(a) forming a contact between a reaction surface on a reaction substrate and an array of open microfluidic channels on a channel substrate, wherein the reaction surface comprises an array of lines of a first plurality of samples; and,
(b) introducing a second plurality of sample solutions into the microfluidic channels, wherein a sample solution contacts the reaction surface along a contact line formed between the reaction surface and each microfluidic channel, and wherein each contact line intersects each line of the first plurality of samples on the reaction surface, thereby contacting each member of the first plurality of samples with each member of the second plurality of samples.

41-43. (canceled)

44. A method of connecting a matrix of sample wells to an array of microfluidic channels, the method comprising the step of:
a) contacting a first surface of a matrix of sample wells to a first surface of a transfer plate, wherein a plurality of wells of said matrix is in fluid connection with a plurality of first channels on said transfer plate; and,
b) contacting a second surface of the transfer plate to a first surface of an array of microfluidic channels, wherein a plurality of microfluidic channels of said array is in fluid connection with a plurality of second channels on said transfer plate, and wherein said plurality of first channels is in fluid connection with said plurality of second channels.

45-53. (canceled)

54. An interface comprising:
a guide adapted to align a tip of a pipette toward a target within a well of a multi-well array; and
a retainer adapted to hold the tip of the pipette in alignment with the target.

55-68. (canceled)

69. A method of placing solution from a pipette tip into a target within a well of a multi-well plate, the method comprising:
guiding the pipette tip toward a position of alignment with the target with a guide mated with the multi-well array;
retaining the pipette tip in the position of alignment with a retainer; and
flowing the solution from the pipette tip and into the target.

70. The method of claim 69, wherein guiding comprises guiding the pipette tip with a funnel shaped portion of a guide plate.

71. The method of claim 70, wherein the guide plate is adapted to mate with the multi-well array and has a plurality of funnel shaped portions each to guide a pipette tip toward a corresponding target.

72. The method of claim 71, wherein the guide plate has a funnel shaped portion corresponding to each well of the multi-well array.

73. The method of claim 72, wherein the target includes an aperture disposed within each well of the multi-well array.

74-115. (canceled)
